(19) United States
(12) Patent Application Publication
(10) Pub. No.: US 2020/0347136 A1
(43) Pub. Date: Nov. 5, 2020

(54) CONSTRAINED CYCLIC PEPTIDES AS INHIBITORS OF THE CD2:CD58 PROTEIN-PROTEIN INTERACTION FOR TREATMENT OF DISEASES AND AUTOIMMUNE DISORDERS

(71) Applicant: Board of Supervisors for the University of Louisiana System, Monroe, LA (US)

(72) Inventor: Seetharama JOIS, West Monroe, LA (US)

(21) Appl. No.: 16/865,295

(22) Filed: May 1, 2020

Related U.S. Application Data
(60) Provisional application No. 62/841,259, filed on May 1, 2019.

(51) Int. Cl. C07K 16/28

(52) U.S. Cl. CPC C07K 16/2824

(57) ABSTRACT

Chemicals and methods of treating autoimmune/inflammatory diseases, comprising administering to a patient the functional groups of a CD58-targeted peptide grafted onto a cyclic scaffold. An additional embodiment includes wherein one or more of the functional groups of the peptide bind to CD58. An additional embodiment includes wherein the scaffold is chosen from the group of sunflower trypsin inhibitor (SFTI) and theta-defensins.

### CROSS REFERENCE TO RELATED APPLICATIONS/PRIORITY

The present invention claims priority to United States Provisional Patent Application No. 62/841,259 filed May 1, 2019, which is incorporated by reference into the present disclosure as if fully restated herein. Any conflict between the incorporated material and the specific teachings of this disclosure shall be resolved in favor of the latter. Likewise, any conflict between an art-understood definition of a word or phrase and a definition of the word or phrase as specifically taught in this disclosure shall be resolved in favor of the latter.

### STATEMENT REGARDING FEDERALLY SPONSORED RESEARCH OR DEVELOPMENT

This invention was made with government support under Grant No. under Grant 1R15CA188225-01A1 (SDJ) awarded by the NCI/National Institutes of Health. The government has certain rights in the invention.

### BACKGROUND

Transient cell-cell interactions and communications play a key role in the immune system and are mediated by specific complementary sets of cell surface proteins. One such interaction is the protein-protein interaction between the integral membrane glycoproteins CD2 and CD58 (also called leukocyte function associated antigen-3, LFA-3), which modulates cell adhesion between T cells (CD2) and epithelial cells (CD58/CD48 in rodents). CD2 is expressed on both CD4+ and CD8+ T cells and is up-regulated upon T cell activation, whereas CD58 is overexpressed by antigen presenting cells in autoimmune diseases.

### SUMMARY

Wherefore, it is an object of the present invention to overcome the above mentioned shortcomings and drawbacks associated with the current technology.

The interaction between the cell-cell adhesion proteins CD2 and CD58 plays a crucial role in lymphocyte recruitment to inflammatory sites, and inhibitors of this interaction have potential as immunomodulatory drugs in autoimmune diseases. Peptides from the CD2 adhesion domain were designed to inhibit CD2:CD58 interactions. To improve the stability of the peptides, β-sheet epitopes from the CD2 region implicated in CD58 recognition were grafted into the cyclic peptide frameworks of sunflower trypsin inhibitor. The designed multicyclic peptides were evaluated for their ability to modulate cell-cell interactions in three different cell adhesion assays, with two candidates, SFTI-a and SFTI-a1, showing potent activity in the nanomolar range (IC₅₀: 51 nM and 22 nM, respectively). Both, SFTI-a and SFTI-a1 exist in multiple conformations in solution. Locking SFTI-a1 in beta conformation resulted in increased inhibition of cell-cell adhesion. The inventors' results evidence that cyclic peptides from natural sources are promising scaffolds for modulating protein-protein interactions that are typically difficult to target with small-molecule compounds.

One embodiment of the presently disclosed invention generally relates to the design and synthesis of peptides that inhibit CD2:CD58 interactions and more specifically to peptides that bind the CD58 receptor to prevent CD2:CD58 interactions, and the treatment of diseases with such peptides.

The inventors are aware that the interaction between CD2 and CD58 has significant importance in autoimmune diseases, such as rheumatoid arthritis (RA). Thus, inhibiting the CD2:CD58 protein-protein interaction is an attractive approach for treating autoimmune disorders. The presently disclosed invention discloses SFTI-a1 and SFTI-DBF, grafted CD2 peptidomimetics that target the CD58 receptor, as novel potent immune modulators through targeting CD2:CD58 interactions.

SFTI-a1
Cyclo(CKSAPPSCAYDGDD)
SFTI-DBF
Cyclo(CKSA-DBF-SCAYDGDD)

Single letter code is used for amino acid representation. Capital letters refer to L-amino acid (P is L-proline). DBF is dibenzofuran. Disulfide bond is indicated by underline. These compounds could be used to treat RA and other immune-related diseases.

The invention further relates to novel chemicals and methods of treating autoimmune/inflammatory diseases, comprising administering to a patient the functional groups of a CD58-targeted peptide grafted onto a cyclic scaffold. An additional embodiment includes wherein one or more of the functional groups of the peptide bind to CD58. An additional embodiment includes wherein the scaffold is chosen from the group of sunflower trypsin inhibitor (SFTI) and theta-defensins.

An additional embodiment wherein the targeted peptide is chosen from the group of:

Peptide 6
Cyclo(SIYDpPDDIK)
SFTI-a
Cyclo(CKASAPPSCYDGDD)
SFTI-a1
Cyclo(CKSAPPSCAYDGDD)
SFTI-DBF
Cyclo(CKSA-DBF-SCAYDGDD)
SFTI-wt (control)
Cyclo(CTKSIPPICFPDGR)
Control
KGKTDAISVKAI-NH2

Single letter code is used for amino acid representation. Capital letters refer to L-amino acid, small letter refers to D-amino acid (P is L-proline, p is D-proline). Cyclo indicates a cyclic compound. DBF is dibenzofuran. Disulfide bond is indicated by underline.

An additional embodiment includes wherein the peptide sequence freely changes conformations or is locked into beta conformation. An additional embodiment includes wherein the peptide sequence is locked into beta conformation through incorporation of 1,4 substituted triazole or substitution of Pro-Pro with dibenzofuran.

An additional embodiment includes wherein the immune disease is chosen from the group of rheumatoid arthritis, psoriatic arthritis, plaque psoriasis, ankylosing spondylitis, Crohn's disease and ulcerative colitis. An additional embodiment includes wherein the cells associated with the immune disease are CD2-positive. An additional embodiment includes wherein the cells are associated with the immune disease are CD58-positive. An additional embodiment includes combining with a pharmacologically effective amount of one or more immune therapy agents from the group of etanercept, adalimumab, infliximab, certolizumab pegol, and golimumab. An additional embodiment wherein the administration route is oral, intravenous, or inhalation. An additional embodiment wherein the peptide is administered with pharmaceutically acceptable excipients.

Among the lung cancers, approximately 85% are histologically classified as non-small-cell lung cancer (NSCLC), a leading cause of cancer deaths worldwide. Epidermal growth factor receptors (EGFRs) are known to play a crucial role in lung cancer. HER2 overexpression is detected by immunohistochemistry in 13%-20% of NSCLC samples, and strong expression is found in only 2%-4%. HER2 amplification is considered an alternative mechanism for the development of resistance to EGFR-targeted tyrosine kinase inhibitor (TKI) therapy. EGFRs have been targeted with three generations of TKIs, and drug resistance has become a major issue; HER2 dimerization with EGFR also plays a major role in the development of resistance to TKI therapy. We have designed grafted peptides to bind to the HER2 extracellular domain (ECD) and inhibit protein-protein interactions of EGFR:HER2 and HER2:HER3. A sunflower trypsin inhibitor (SFTI) template was used to graft a peptidomimetic compound that exhibits antiproliferative activity with an IC₅₀ value of 18 nM in NSCLC cell lines. Among several grafted peptides, SFTI-G5 exhibited antiproliferative activity in HER2-positive NSCLC cell lines with an IC₅₀ value of 0.073 μM. SFTI-G5 was shown to bind to HER2 and inhibit EGFR:HER2 and HER2:HER3 dimerization and also inhibit the phosphorylation of HER2 and downstream signaling proteins such as Akt. As a proof-of-concept, the in vivo activity of SFTI-G5 was evaluated in two NSCLC mouse models. SFTI-G5 was able to inhibit tumor growth in both models. Furthermore, SFTI-G5 was shown to inhibit EGFR dimerization in tissue samples obtained from in vivo models. These grafted peptides show chemical and thermal stability and, hence, can be used as novel dual inhibitors of EGFR dimerization in NSCLC.

In some embodiments, the therapeutic, or a pharmaceutically acceptable salt, solvate, or prodrug thereof, is administered as a pharmaceutical composition that further includes a pharmaceutically acceptable excipient.

In some embodiments, administration of the pharmaceutical composition to a human results in a peak plasma concentration of the therapeutic between 0.05 μM-10 μM (e.g., between 0.05 μM-5 μM).

In some embodiments, the peak plasma concentration of the therapeutic is maintained for up to 14 hours. In other embodiments, the peak plasma concentration of the therapeutic is maintained for up to 1 hour.

In other embodiments, the therapeutic is administered at a dose that is between 0.05 mg-5 mg/kg weight of the human.

In certain embodiments, the pharmaceutical composition is formulated for oral administration.

In other embodiments, the pharmaceutical composition is formulated for extended release.

In still other embodiments, the pharmaceutical composition is formulated for immediate release.

In some embodiments, the pharmaceutical composition is administered concurrently with one or more additional therapeutic agents for the treatment or prevention of the disease or condition.

In some embodiments, the therapeutic, or a pharmaceutically acceptable salt, solvate, or prodrug thereof, is administered as a pharmaceutical composition that further includes a pharmaceutically acceptable excipient.

In some embodiments, administration of the pharmaceutical composition to a human results in a peak plasma concentration of the therapeutic between 0.05 μM-10 μM (e.g., between 0.05 μM-5 μM).

In some embodiments, the peak plasma concentration of the therapeutic is maintained for up to 14 hours. In other embodiments, the peak plasma concentration of the therapeutic is maintained for up to 1 hour.

In other embodiments, the therapeutic is administered at a dose that is between 0.05 mg-5 mg/kg weight of the human.

In certain embodiments, the pharmaceutical composition is formulated for oral administration.

In other embodiments, the pharmaceutical composition is formulated for extended release.

In still other embodiments, the pharmaceutical composition is formulated for immediate release.

As used herein, the term “delayed release” includes a pharmaceutical preparation, e.g., an orally administered formulation, which passes through the stomach substantially intact and dissolves in the small and/or large intestine (e.g., the colon). In some embodiments, delayed release of the active agent (e.g., a therapeutic as described herein) results from the use of an enteric coating of an oral medication (e.g., an oral dosage form).

The term an “effective amount” of an agent, as used herein, is that amount sufficient to effect beneficial or desired results, such as clinical results, and, as such, an “effective amount” depends upon the context in which it is being applied.

The terms “extended release” or “sustained release” interchangeably include a drug formulation that provides for gradual release of a drug over an extended period of time, e.g., 6-12 hours or more, compared to an immediate release formulation of the same drug. Preferably, although not necessarily, results in substantially constant blood levels of a drug over an extended time period that are within therapeutic levels and fall within a peak plasma concentration range that is between, for example, 0.05-10 μM, 0.1-10 μM, 0.1-5.0 μM, or 0.1-1 μM.

As used herein, the terms “formulated for enteric release” and “enteric formulation” include pharmaceutical compositions, e.g., oral dosage forms, for oral administration able to provide protection from dissolution in the high acid (low pH) environment of the stomach. Enteric formulations can be obtained by, for example, incorporating into the pharmaceutical composition a polymer resistant to dissolution in gastric juices. In some embodiments, the polymers have an optimum pH for dissolution in the range of approx. 5.0 to 7.0 (“pH sensitive polymers”). Exemplary polymers include methacrylate acid copolymers that are known by the trade name Eudragit® (e.g., Eudragit® L100, Eudragit® S100, Eudragit® L-30D, Eudragit® FS 30D, and Eudragit® L100-55), cellulose acetate phthalate, cellulose acetate trimellitiate, polyvinyl acetate phthalate (e.g., Coateric®), hydroxyethylcellulose phthalate, hydroxypropyl methylcellulose phthalate, or shellac, or an aqueous dispersion thereof. Aqueous dispersions of these polymers include dispersions of cellulose acetate phthalate (Aquateric®) or shellac (e.g., MarCoat 125 and 125N). An enteric formulation reduces the percentage of the administered dose released into the stomach by at least 50%, 60%, 70%, 80%, 90%, 95%, or even 98% in comparison to an immediate release formulation. Where such a polymer coats a tablet or capsule, this coat is also referred to as an “enteric coating.”

The term “immediate release” includes where the agent (e.g., therapeutic), as formulated in a unit dosage form, has a dissolution release profile under in vitro conditions in which at least 55%, 65%, 75%, 85%, or 95% of the agent is released within the first two hours of administration to, e.g., a human. Desirably, the agent formulated in a unit dosage has a dissolution release profile under in vitro conditions in which at least 50%^{, 65}%^{, 75}%^{, 85}%^{, 90}%, or 95% of the agent is released within the first 30 minutes, 45 minutes, or 60 minutes of administration.

The term “pharmaceutical composition,” as used herein, includes a composition containing a compound described herein (or any pharmaceutically acceptable salt, solvate, or prodrug thereof), formulated with a pharmaceutically acceptable excipient, and typically manufactured or sold with the approval of a governmental regulatory agency as part of a therapeutic regimen for the treatment of disease in a mammal.

Pharmaceutical compositions can be formulated, for example, for oral administration in unit dosage form (e.g., a tablet, capsule, caplet, gelcap, or syrup); for topical administration (e.g., as a cream, gel, lotion, or ointment); for intravenous administration (e.g., as a sterile solution free of particulate emboli and in a solvent system suitable for intravenous use); or in any other formulation described herein.

A “pharmaceutically acceptable excipient,” as used herein, includes any ingredient other than the compounds described herein (for example, a vehicle capable of suspending or dissolving the active compound) and having the properties of being nontoxic and non-inflammatory in a patient. Excipients may include, for example: antiadherents, antioxidants, binders, coatings, compression aids, disintegrants, dyes (colors), emollients, emulsifiers, fillers (diluents), film formers or coatings, flavors, fragrances, glidants (flow enhancers), lubricants, preservatives, printing inks, sorbents, suspensing or dispersing agents, sweeteners, or waters of hydration. Exemplary excipients include, but are not limited to: butylated hydroxytoluene (BHT), calcium carbonate, calcium phosphate (dibasic), calcium stearate, croscarmellose, cross-linked polyvinyl pyrrolidone, citric acid, crospovidone, cysteine, ethylcellulose, gelatin, hydroxypropyl cellulose, hydroxypropyl methylcellulose, lactose, magnesium stearate, maltitol, maltose, mannitol, methionine, methylcellulose, methyl paraben, microcrystalline cellulose, polyethylene glycol, polyvinyl pyrrolidone, povidone, pregelatinized starch, propyl paraben, retinyl palmitate, shellac, silicon dioxide, sodium carboxymethyl cellulose, sodium citrate, sodium starch glycolate, sorbitol, starch (corn), stearic acid, stearic acid, sucrose, talc, titanium dioxide, vitamin A, vitamin E, vitamin C, and xylitol.

The term “pharmaceutically acceptable prodrugs” as used herein, includes those prodrugs of the compounds of the present invention which are, within the scope of sound medical judgment, suitable for use in contact with the tissues of humans and animals with undue toxicity, irritation, allergic response, and the like, commensurate with a reasonable benefit/risk ratio, and effective for their intended use, as well as the zwitterionic forms, where possible, of the compounds of the invention.

The term “pharmaceutically acceptable salt,” as use herein, includes those salts which are, within the scope of sound medical judgment, suitable for use in contact with the tissues of humans and animals without undue toxicity, irritation, allergic response and the like and are commensurate with a reasonable benefit/risk ratio. Pharmaceutically acceptable salts are well known in the art. For example, pharmaceutically acceptable salts are described in: Berge et al., J. Pharmaceutical Sciences 66:1-19, 1977 and in Pharmaceutical Salts: Properties, Selection, and Use, (Eds. P. H. Stahl and C. G. Wermuth), Wiley-VCH, 2008. The salts can be prepared in situ during the final isolation and purification of the compounds of the invention or separately by reacting the free base group with a suitable organic or inorganic acid. Representative acid addition salts include acetate, adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, fumarate, glucoheptonate, glycerophosphate, hemisulfate, heptonate, hexanoate, hydrobromide, hydrochloride, hydroiodide, 2-hydroxy-ethanesulfonate, lactobionate, lactate, laurate, lauryl sulfate, malate, maleate, malonate, methanesulfonate, 2-naphthalenesulfonate, nicotinate, oleate, oxalate, palmitate, pamoate, pectinate, persulfate, 3-phenylpropionate, phosphate, picrate, pivalate, propionate, stearate, succinate, sulfate, tartrate, thiocyanate, toluenesulfonate, undecanoate, valerate salts, and the like. Representative alkali or alkaline earth metal salts include sodium, lithium, potassium, calcium, magnesium, and the like, as well as nontoxic ammonium, quaternary ammonium, and amine cations, including, but not limited to ammonium, tetramethylammonium, tetraethylammonium, methylamine, dimethylamine, trimethylamine, triethylamine, ethylamine, and the like.

The terms “pharmaceutically acceptable solvate” or “solvate,” as used herein, includes a compound of the invention wherein molecules of a suitable solvent are incorporated in the crystal lattice. A suitable solvent is physiologically tolerable at the administered dose. For example, solvates may be prepared by crystallization, recrystallization, or precipitation from a solution that includes organic solvents, water, or a mixture thereof. Examples of suitable solvents are ethanol, water (for example, mono-, di-, and tri-hydrates), N-methylpyrrolidinone (NMP), dimethyl sulfoxide (DMSO), N,N′-dimethylformamide (DMF), N,N′-dimethylacetamide (DMAC), 1,3-dimethyl-2-imidazolidinone (DMEU), 1,3-dimethyl-3,4,5,6-tetrahydro-2-(1H)-pyrimidinone (DMPU), acetonitrile (ACN), propylene glycol, ethyl acetate, benzyl alcohol, 2-pyrrolidone, benzyl benzoate, and the like. When water is the solvent, the solvate is referred to as a “hydrate.”

The term “prevent,” as used herein, includes prophylactic treatment or treatment that prevents one or more symptoms or conditions of a disease, disorder, or conditions described herein. Treatment can be initiated, for example, prior to (“pre-exposure prophylaxis”) or following (“post-exposure prophylaxis”) an event that precedes the onset of the disease, disorder, or conditions. Treatment that includes administration of a compound of the invention, or a pharmaceutical composition thereof, can be acute, short-term, or chronic. The doses administered may be varied during the course of preventive treatment.

The term “prodrug,” as used herein, includes compounds which are rapidly transformed in vivo to the parent compound of the above formula. Prodrugs also encompass bioequivalent compounds that, when administered to a human, lead to the in vivo formation of therapeutic. A thorough discussion is provided in T. Higuchi and V. Stella, Pro-drugs as Novel Delivery Systems, Vol. 14 of the A.C.S. Symposium Series, and Edward B. Roche, ed., Bioreversible Carriers in Drug Design, American Pharmaceutical Association and Pergamon Press, 1987, each of which is incorporated herein by reference. Preferably, prodrugs of the compounds of the present invention are pharmaceutically acceptable.

As used herein, and as well understood in the art, “treatment” includes an approach for obtaining beneficial or desired results, such as clinical results. Beneficial or desired results can include, but are not limited to, alleviation or amelioration of one or more symptoms or conditions; diminishment of extent of disease, disorder, or condition; stabilized (i.e. not worsening) state of disease, disorder, or condition; preventing spread of disease, disorder, or condition; delay or slowing the progress of the disease, disorder, or condition; amelioration or palliation of the disease, disorder, or condition; and remission (whether partial or total), whether detectable or undetectable. “Treatment” can also mean prolonging survival as compared to expected survival if not receiving treatment. As used herein, the terms “treating” and “treatment” can also include delaying the onset of, impeding or reversing the progress of, or alleviating either the disease or condition to which the term applies, or one or more symptoms of such disease or condition.

The term “unit dosage forms” includes physically discrete units suitable as unitary dosages for human subjects and other mammals, each unit containing a predetermined quantity of active material calculated to produce the desired therapeutic effect, in association with any suitable pharmaceutical excipient or excipients.

As used herein, the term “plasma concentration” includes the amount of therapeutic present in the plasma of a treated subject (e.g., as measured in a rabbit using an assay described below or in a human).

Various objects, features, aspects, and advantages of the present invention will become more apparent from the following detailed description of preferred embodiments of the invention, along with the accompanying drawings in which like numerals represent like components. The present invention may address one or more of the problems and deficiencies of the current technology discussed above. However, it is contemplated that the invention may prove useful in addressing other problems and deficiencies in a number of technical areas. Therefore the claimed invention should not necessarily be construed as limited to addressing any of the particular problems or deficiencies discussed herein.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The accompanying drawings, which are incorporated in and constitute a part of the specification, illustrate various embodiments of the invention and together with the general description of the invention given above and the detailed description of the drawings given below, serve to explain the principles of the invention. It is to be appreciated that the accompanying drawings are not necessarily to scale since the emphasis is instead placed on illustrating the principles of the invention. The invention will now be described, by way of example, with reference to the accompanying drawings in which:

FIG. 1 shows cell adhesion is important for immune response. Schematic diagram of signals involved in immune response and inhibition of immune response. A) Recognition of antigens on APC by T cells according to the two-signal hypothesis. i) T-cell activation requires recognition of an antigen by the T-cell receptor (TCR) (Signal 1) and a concomitant signal provided by adhesion/co-stimulatory molecules (Signal 2) to achieve full activation of T cells. Bretscher et al., Proc Natl Acad Sci USA, 96(1), 185-190 (1999); Chen et al., Nature reviews. Immunology, 13(4), 227-242 (2013). ii) Co-stimulatory molecules or adhesion molecules can be targeted with cyclic peptides and grafted cyclic peptides or antibodies to inhibit the protein-protein interaction between co-stimulatory molecules resulting in suppression of particular immune response. B) Schematic diagram showing the importance of adhesion molecules CD2-CD58 in immune response and protein-protein interactions of CD2-CD58 and other costimulatory molecules. Several protein molecules on the cell surface help to hold cells together during immune response generation (like Velcro®). CD2-CD58 molecules make the first move. CD2 and CD58 are highly expressed in RA patients.

FIG. 2 shows designs of peptides from CD2 protein and grafting of CD2 peptide to SFTI framework. i) Crystal structure of CD2-CD58 complex shown in secondary structure (Protein data bank (PDB) ID: 1QA9). Adhesion domain of CD2 protein with F and C beta-strands interacting with CD58 protein. ii) Amino acids in F and C strands and iii) design of peptide from conformational constraints. iv) Grafting the F and C strand amino acids onto sunflower trypsin inhibitor (SFTI) with beta-strand structures. v) Grafted peptide for PPI inhibition SFTI-a with IC₅₀ 51 nM. vi) New grafted peptide SFTI-a1 with IC₅₀ 22 nM. Enzyme binding loop indicated in red box and Ser-Lys and Ser-Ala-Lys sequence indicated in blue circles.

FIG. 3 shows CD2 peptide reduces cell adhesion, arthritis, and collagen antibody levels. A) Inhibition of E-rosette formation by synthetic peptide 6 derived from CD2 protein. Peptides were added to AET-treated sheep red blood cells (SRBC) (expressing CD58 protein) first, and Jurkat cells (expressing CD2 protein) labeled with BCECF were added later. Cells with five or more SRBC bound were counted as rosettes. Values are percent inhibition of peptide-treated cells and are expressed as the mean of three experimental values. Error bars represent standard error of the mean (SEM). B) Suppression of arthritis by peptide 6 in collagen-induced arthritis. The bar diagram depicts suppression of collagen-induced arthritis 40 days postimmunization. Mice treated with peptide 6 at concentrations of 0.25, 0.5, and 1 mg/kg showed statistically significant differences in arthritis severity (*p<0.05) from days 42 to 46 compared with the control peptide. Scoring was carried out according to the published procedure (0-4) as described in the text for all four limbs with a maximum score of 16. Plots depict mean scores in each group. Only arthritic mice were used for severity index. Values represent the mean SEM. C) Histopathology analysis of sample from paws from normal, arthritic, and treated DBA/1 mice. Sections of paws from 6 mice were chosen, and representative sections were chosen for final analysis. i) Normal phalangeal joints of hind foot. Cartilaginous surfaces are intact and smooth (arrow heads), and joint space is clear (*). H & E staining. Original magnification 200×. ii) Arthritis hind paw with severe (grade 4) arthritis of phalangeal joint. Joint is swollen. Marked cartilaginous erosion of joint surface and pannus circumscribing the phalanges. H & E staining. Original magnification 200×. iii, iv) Arthritis hind paw with severe (grade 4) erosion of cartilage and bone resorption (arrows) of phalanges. The connective tissue (*) of the dermis, hypodermis, and peristeum is mildly edematous and infiltrated with neutrophils, mixed mononuclear inflammatory cells, and fibroblasts. H & E staining. Original magnification 100×. v) Hind paw of mouse treated with peptide 6 (0.5 mg/kg). Joint space is clear (*) with normal cartilage interface. Connective tissue surrounding joint is only very minimally infiltrated with mixed inflammatory cells. H & E staining. Original magnification 100×. D) Reduction in circulating anti-CII Ab titer in serum of mice with collagen-induced arthritis. N=6. Peptide 6 was injected intravenously (i.v.) at 0.5 and 1 mg/kg. Relative levels of anti-CII Ab titer were observed with peptide 6-treated mice compared with control peptide and control groups with no treatment. Values represent the mean SEM. Statistical analysis indicated that p<0.05 for peptide 6 treated mice compared with control and control peptide-treated mice (Gokhale et al. J. Med. Chem. 2011, 54: 5307-5319; Gokhale et al. Chem. Biol. Drug Des. 2013, 82: 106-118).

FIG. 4 shows designs of constrained grafted peptides using CD2 protein sequence. i) Fragment of CD2 protein that binds to CD58. This fragment can be used to block CD2-CD58 interaction and immunomodulation. ii) sunflower-trypsin inhibitor frame work. iii) Grafted peptides designed based on SFTI-1 and CD2 fragment. Pro-Pro sequence is shown in rectangle. iv) SFTI-a was modified using alanine scanning to generate SFTI-a1. v) peptide was conformationally constrained using dibenzofuran moiety. Instead of Pro-Pro sequence in SFTI-a, dibenzofuran (DBF) was used to generate the peptidomimetic SFTI-DBF.

FIG. 5 shows CD2 peptidomimetic locked in beta conformation, SFTI-DBF, is more effective at inhibiting cell adhesion than unlocked CD2 peptidomimetic. Table of stable peptides designed based on sunflower trypsin inhibitor (SFTI) and their cell adhesion inhibition activities in a model system. OVCAR-3 and HFLS-RA cells express CD58. Jurkat T cells express CD2 protein. Inhibition of adhesion between Jurkat and OVCAR-3/HFLS-RA cells was evaluated. Cell adhesion inhibition is represented as IC₅₀ value

FIG. 6 shows CD2 peptide exists in multiple conformations prior to conformation locking. Two dimensional 1H NMR Total Correlation Spectroscopy (TOCSY) spectra of A) SFTI-a showing a large number of peaks for the peptide sequence, indicating at least 3 to 4 resonances for each amino acid. Thus, SFTI-a exhibits at least 3 conformations in solution. B) SFTI-DBF (dibenzofuran) showing one resonance peak for each amino acid in the spectra indicating one major conformer of the peptide in solution. SFTI-a and SFTI-DBF were dissolved in deuterated dimethyl sulfoxide (DMSO-d6) for NMR data collection.

FIG. 7 shows CD2 peptidomimetic locked in beta conformation, SFTI-DBF, inhibits cell adhesion at low nanomolar concentrations. In a cell adhesion assay where Jurkat cells express CD2 and OVCAR-3 and HFLS-RA cells express CD58, dose response of SFTI-DBF showed A) IC₅₀ of 0.61 nM for Jurkat/OVCAR-3 cells and B) IC₅₀ of 3 nM for Jurkat/HFLS-RA cells.

FIG. 8 shows A) A schematic diagram of EGFR dimerization for downstream signaling in NSCLC showing the target site of SFTI-G5, a grafted peptide that targets EGFR:HER2 and HER3:HER2 dimerization. Design concept of grafted sunflower trypsin inhibitor (SFTI) peptide based on B) compound 18 that is known to bind to HER2 ECD domain IV and inhibit EGFR:HER2, HER2:HER3 dimerization. C) Sunflower trypsin inhibitor-1 (SFTI-1) peptide. D) Grafting of pharmacophore from compound 18 to SFTI framework, resulting in a grafted peptide. Grafted residues are shown in red. X=3-amino 3(1-napthyl) propionic acid (Anapa).

FIG. 9 shows designs of grafted peptides. A) Amino acids and beta-amino acids from compound 18 were grafted onto B) SFTI framework to design different possible grafted peptides. C) SFTI-G1 to G6 peptides. Amino acids from compound 18 are highlighted in yellow and those from SFTI are shown in pink. Anapa: 3-amino 3(1-napthyl) propionic acid.

FIG. 10 shows A) SPR analysis of binding of SFTI-G5 with HER2 protein. Binding to ECD protein at different concentrations. Kinetics of association and dissociation are shown. Inset saturation curve for binding. B) SPR analysis of EGFR:HER2 dimerization and its inhibition by SFTI-G5. EGFR was immobilized on CM5-chip and HER2 was added as analyte. Binding of HER2 to EGFR is shown in red. Different concentrations of SFTI-G5 along with constant concentration of HER2 were added. Relative RU was decreased with increased concentration of SFTI-G5, indicating inhibition of protein-protein interaction of ECD of EGFR: HER2. C) EGFR:HER2 and HER2:HER3 dimerization in I) Calu-3 and II) A549 cells and inhibition of dimerization by SFTI-G5 as shown by PLA assay. I) EGFR:HER2 (A) and HER2:HER3 (E) dimerization in Calu-3 cells and its inhibition by SFTI-G5 at 0.5 and 1 (C & D, G & H). Control represents cells without the peptide and without one of the antibodies (B & E). II) EGFR: HER2 (A) and HER2:HER3 (E) dimerization in A549 cells and its inhibition by SFTI-G5 at 0.5 and 1 (C & D, G & H). Control represents cells without the peptide and without one of the antibodies (F). Note the decrease in the number of red dots in the treatment group, suggesting the inhibition of dimerization. Nucleus was stained with DAPI. III) Quantification of PLA assay fluorescence intensity. 3-6 different fields were chosen, and intensity of red fluorescence was measured using ImageJ software. Notice that, upon addition of SFTI-G5, red fluorescence was decreased, indicating inhibition of dimers. Scale 20 μm. Magnification 40×. *p<0.05, ** p<0.01. Mean±SEM. D) SFTI-G5 inhibits the phosphorylation of HER2 and Akt. Western blot analysis of phosphorylation of HER2 and Akt after treatment of vehicle, SFTI-G5 (1 μM), and Lapatinib (2 μM) for 40 h in A549 cells. The visualization of GAPDH was used to ensure equal sample loading in each lane. I) SFTI-G5 (1 μM) inhibits the phosphorylation of HER2 kinase and Akt compared to control in A549 cells as shown by Western blot. II) Quantification of Western blot images using densitometry. *p<0.05 *** P<0.001. Mean±SEM.

FIG. 11 shows Xenograft Lung cancer development and treatment by SFTI-G5. 5×10⁶ Calu-3 cells suspended in serum-free medium were subcutaneously inoculated in left flank region of mice to induce lung cancer (Foxn1-nude). Peptide (8 mg/kg) was administered via intratumoral injection three times a week after 10 days, Pertuzumab was injected with a loading dose of 12 mg/kg and then 6 mg/kg as maintenance dose intraperitonially once a week, Trastuzumab and pertuzumab combination was injected at a loading dose of 12 mg/kg and then 6 mg/kg as maintenance dose once a week. A) Timeline of experiment; B) in vivo antitumorigenic activity of SFTI-G5; the tumor volume (V) was calculated as V=(L×W²)/2, where L was the length and W was the width of tumors. SFTI-G5(8 mg/kg) (square marks) delayed tumor growth in athymic nude mice significantly compared to the control group without any treatment (circles). For comparison, pertuzumab (up triangle, blue) and trastuzumab and pertuzumab combination (down triangle, red) also shown (with standard errors for n=6). Statistical analysis indicated that there was a significant difference between control and SFTI-G5 (*** p<0.001) after 21 days. C) PPI inhibition assessed by PLA on tumor tissue sample. C) EGFR:HER2 PPI and its inhibition in vehicle control, SFTI-G5-treated and trastuzumab- and pertuzumab-treated mice groups. Magnification 10×, scale 100 μm, D) Western blot analysis of tumor sections of control group, mice treated with SFTI-G5 group, pertuzumab group, and trastuzumab and pertuzumab combination group. Tumor sections were homogenized, and protein was extracted and subjected to Western blot. SFTI-G5 showed a decrease in phosphorylation of HER2 kinase. Statistical analysis showed that there was significant reduction of phosphorylation HER2 protein in SFTI-G5 group. (** p<0.01). Mean±SEM.

FIG. 12 shows A) Lung cancer development and treatment by SFTI-G5. A549 Red-FLuc Luciferase transfected cells were injected to induce lung cancer in mice (Foxn1-nude). One week after injection of cells, mice were monitored for lung cancer development using luciferin imaging. Peptide was administered via IV twice a week after 2 weeks. Relative luminescence intensity (total photon flux/sec) plotted for 4 animals and controls. B) Representative images: In the control group (Row 1) lung cancer tumor growth increased in 5 weeks. In animals that were treated with SFTI-G5 (Rows 2 and 3) via IV injection (6 mg/kg, 100 μL), tumor growth was reduced compared to the control group as shown by reduction in bioluminescence of tumor in weeks 4 and 5. C) PPI inhibition assessed by PLA on lung tumor tissue. C) EGFR:HER2 PPI and its inhibition in SFTI-G5 treated mice. D) HER3: HER2 PPI and its inhibition by SFTI-G5 treated mice. Note the decrease in red fluorescence in treated mice lung cancer tissue due to inhibition of PPI by SFTI-G5. Magnification 40×. Scale bar for C&D 20 μm. Bottom panel: Histopathology of tumor samples from in vivo study of lung cancer. Scale bar 100 μm. D) Immunogenicity of peptide in animal model. Cells from spleen of mice primed with SFTI-G5 for antigenicity study. BALB/C mice were primed with 6 mg/kg of SFTI-G5 and splenocytes were isolated after 13th day from control and primed mice groups. After harvesting the cells, SFTI-G5 was added at 5 μM concentration. Significance of comparisons were made for ConA versus control group and peptide treatment group. (n=6, **** p<0.0001).

### DETAILED DESCRIPTION

The present invention will be understood by reference to the following detailed description, which should be read in conjunction with the appended drawings. It is to be appreciated that the following detailed description of various embodiments is by way of example only and is not meant to limit, in any way, the scope of the present invention. In the summary above, in the following detailed description, in the claims below, and in the accompanying drawings, reference is made to particular features (including method steps) of the present invention. It is to be understood that the disclosure of the invention in this specification includes all possible combinations of such particular features, not just those explicitly described. For example, where a particular feature is disclosed in the context of a particular aspect or embodiment of the invention or a particular claim, that feature can also be used, to the extent possible, in combination with and/or in the context of other particular aspects and embodiments of the invention, and in the invention generally. The term “comprises” and grammatical equivalents thereof are used herein to mean that other components, ingredients, steps, etc. are optionally present. For example, an article “comprising” (or “which comprises”) components A, B, and C can consist of (i.e., contain only) components A, B, and C, or can contain not only components A, B, and C but also one or more other components. Where reference is made herein to a method comprising two or more defined steps, the defined steps can be carried out in any order or simultaneously (except where the context excludes that possibility), and the method can include one or more other steps which are carried out before any of the defined steps, between two of the defined steps, or after all the defined steps (except where the context excludes that possibility).

The term “at least” followed by a number is used herein to denote the start of a range beginning with that number (which may be a range having an upper limit or no upper limit, depending on the variable being defined). For example “at least 1” means 1 or more than 1. The term “at most” followed by a number is used herein to denote the end of a range ending with that number (which may be a range having 1 or 0 as its lower limit, or a range having no lower limit, depending upon the variable being defined). For example, “at most 4” means 4 or less than 4, and “at most 40% means 40% or less than 40%. When, in this specification, a range is given as “(a first number) to (a second number)” or “(a first number)-(a second number),” this means a range whose lower limit is the first number and whose upper limit is the second number. For example, 25 to 100 mm means a range whose lower limit is 25 mm, and whose upper limit is 100 mm. The embodiments set forth the below represent the necessary information to enable those skilled in the art to practice the invention and illustrate the best mode of practicing the invention. In addition, the invention does not require that all the advantageous features and all the advantages need to be incorporated into every embodiment of the invention.

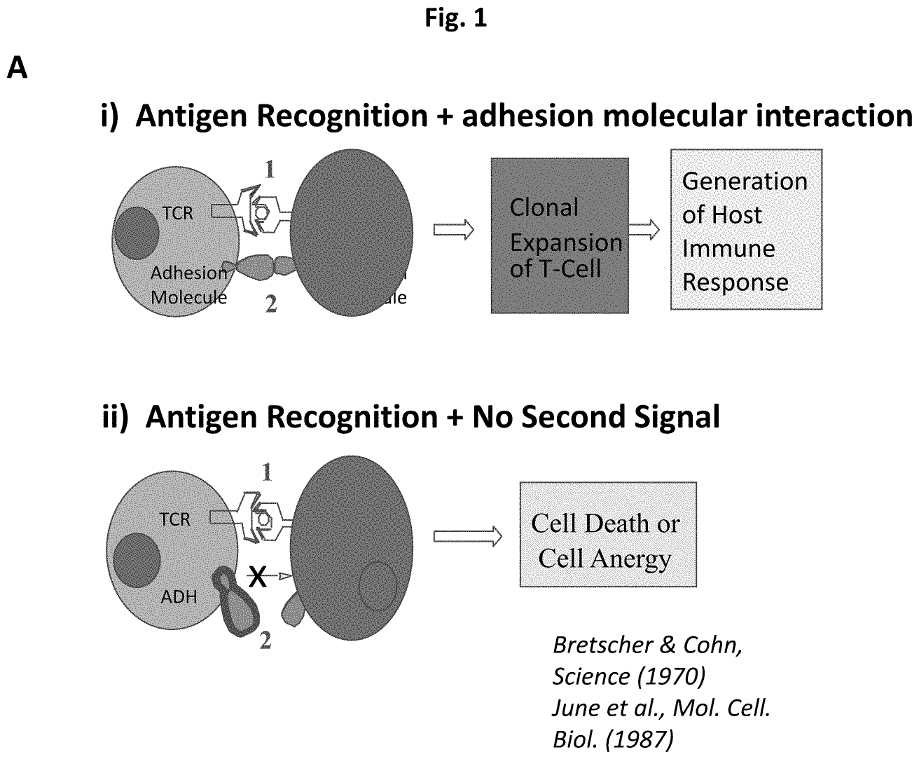

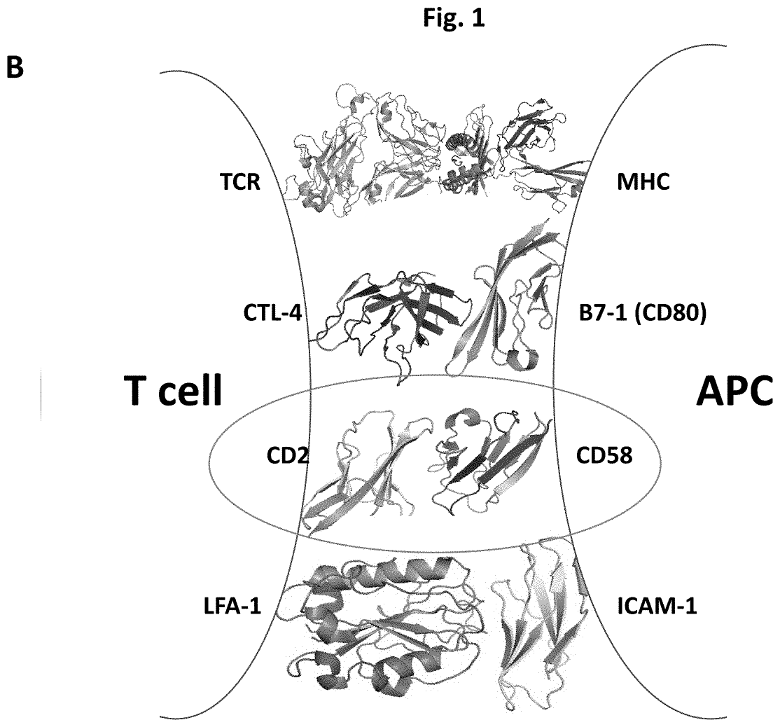

Turning now to FIGS. 1-12, a brief description concerning the various components of the present invention will now be briefly discussed.

Significance of CD2:CD58 Interactions. Protein-protein interactions (PPI) control many physiological processes in the body and any deregulation of PPI results in disease states. The PPI between CD2 on T cells and CD58 on antigen-presenting cells (APC) is important in the early stage of immune response. The two-signal hypothesis proposes that T-cell activation requires recognition of an antigen by the T-cell receptor (TCR) (Signal 1) and a concomitant signal provided by adhesion/co-stimulatory molecules (Signal 2) to achieve full activation of T cells (FIG. 1Ai, ii). Among the adhesion/co-stimulatory molecules, the most abundant are CD2, a transmembrane protein in T cells that binds to its ligand CD58 on APC (CD48 in mice). CD48 has a high degree of homology to CD58 (˜60%) and is similar in 3D structure CD58. Ligation of CD2 on T cells to CD58 on APC facilitates T cell-APC adhesion and is important for the generation of an immune response. This interaction results in the induction of IFN-γ and subsequent regulation of human leukocyte antigen-antigen D related (HLA-DR) complex, intracellular adhesion molecule-1 (CAM-1), and B-7 molecules on APC, causing an amplification of the signal for immune response (FIG. 1B). CD2 and CD58 molecules have been shown to be important in inflammatory diseases, and there is an upregulation of CD58 in inflammatory diseases. Alefacept is a recombinant human CD58-Ig fusion protein that effectively binds to CD2 and prevents CD2 interaction with CD58. It has been successfully used clinically to treat plaque psoriasis. These findings make CD2 and CD58 molecules attractive targets for understanding the mechanism(s) of PPI inhibition in cell adhesion in autoimmune and inflammatory diseases.

CD58 is a cell adhesion molecule with only one known ligand, CD2. It plays a critical role in the facilitation of antigen-specific recognition through interaction with CD2 on T lymphocytes and natural killer cells. Blocking of adhesion/co-stimulatory molecules results in blocking PPI and preventing generation of the immune response. The inventors have successfully designed and evaluated peptides from CD2 protein to block CD2-CD58 interaction in a model system of T cells and epithelial cells. The inventors have generated multicyclic peptides from plant sources for grafting biologically active epitopes from CD2 protein to generate peptides to inhibit PPI of CD2-CD58.

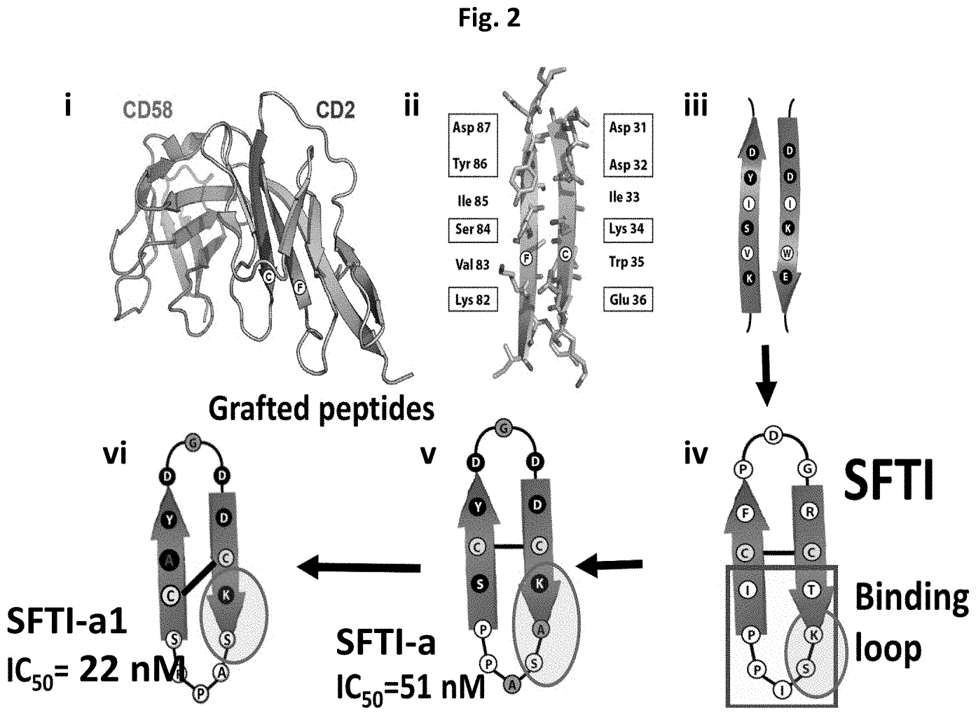

Design and Synthesis of Grafted Peptides. Multicyclic plant-derived peptides (cyclotides), or mini-proteins that have a cyclic structure with disulfide bonds, are resistant to thermal, chemical, and enzymatic degradation, and are orally bioavailable. Generally, these multicyclic peptides do not present any immunogenicity and are amenable to sequence modification. Although antibodies have been targeted to inhibit PPI for cell adhesion molecules, they have limitations in terms of stability, immunogenicity, and formulation. The inventors have designed grafted multicyclic peptides based on a plant peptide sunflower trypsin inhibitor (SFTI) to inhibit CD2:CD58 interaction (FIG. 2). The adhesion domain of CD2 has important residues in F and C strands that participate in hydrogen bonding and hydrophobic interaction with CD58 protein to stabilize protein-protein interactions for cell adhesion (FIG. 2i). The design of the Peptide 6 was based on the structure of the CD2-CD58 complex (PDB ID: 1QA9) (FIG. 2i), as well as on the inventors' previous studies. The CD58 binding domain of CD2 consists of β-strands with charged residues. Peptides designed from β-strands exhibit cell adhesion inhibition activity. Peptide 6 is based on F and C strands of CD2 (FIG. 2ii) and consists of (D)-Pro-Pro for β-turn and Ser84-Asp87 to Asp31-Lys34 residues from CD2 protein sequence (FIG. 2iii). The binding loop in SFTI peptide (FIG. 2iv) contains amino acids Lys-Ser that are resistant to enzymatic degradation. SFTI-a has a Lys-Ala-Ser (FIG. 2v) sequence, whereas SFTI-a1 has Lys-Ser (FIG. 2vi) in the sequence. Retaining this Lys-Ser containing loop is known to maintain enzymatic stability.

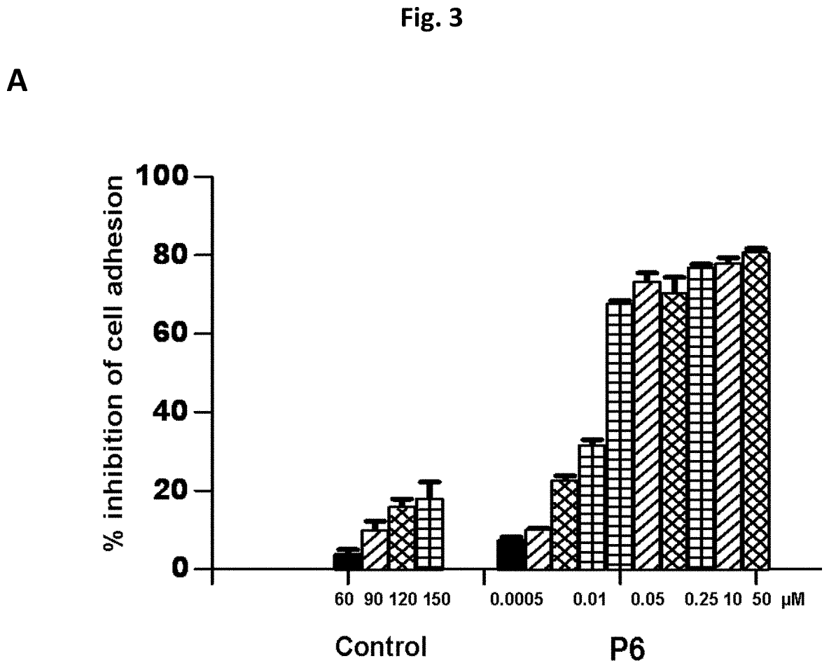

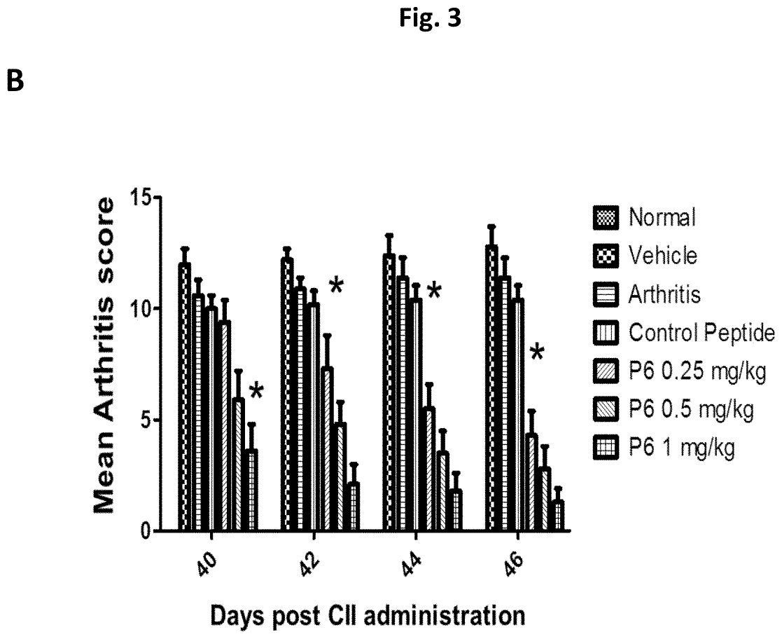

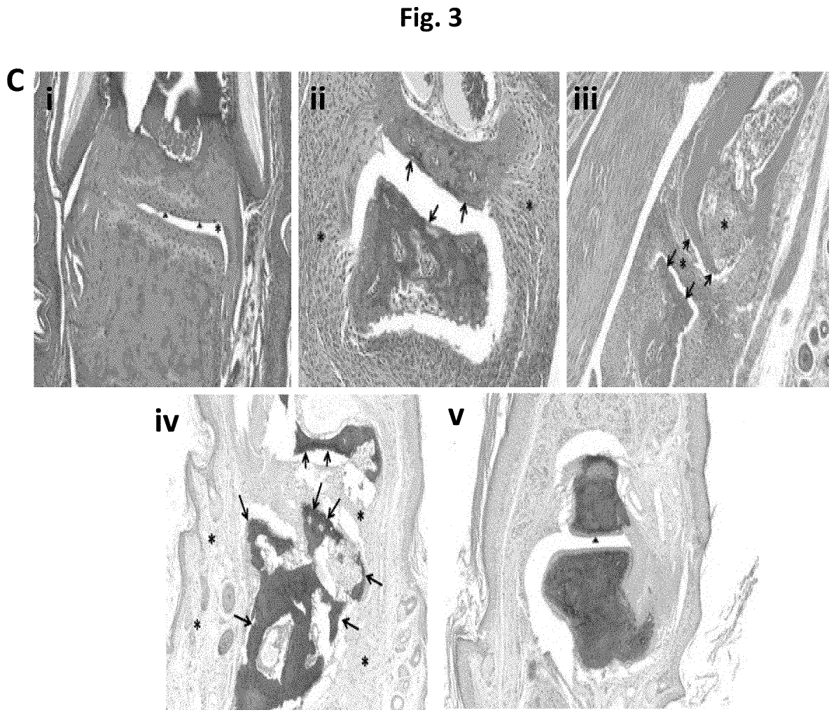

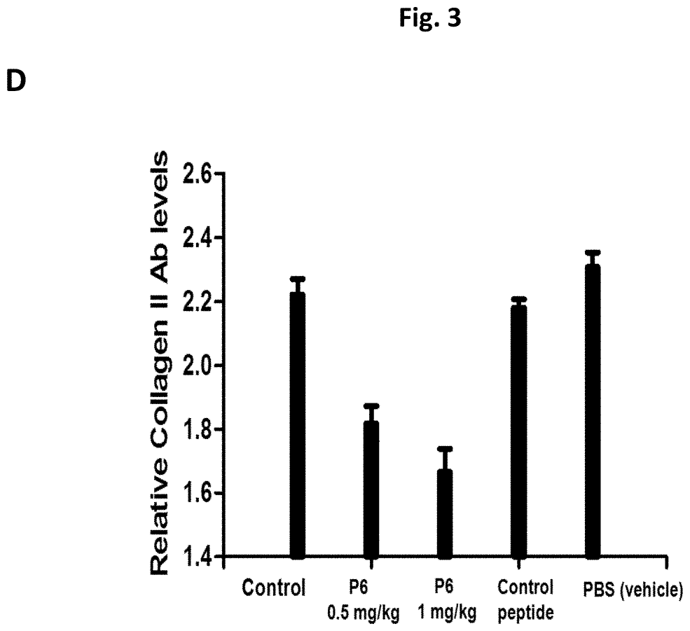

Peptide 6 reduces cell-cell adhesion. The ability of peptides to inhibit cell adhesion was evaluated using E-rosetting assay via interaction between sheep red blood cells (SRBC) expressing CD58 and Jurkat T cells expressing CD2 protein. When these cells are incubated, they adhere to each other. Each Jurkat cell adheres to many sheep red blood cells. Five or more SRBC adhering to a Jurkat cell is counted as positive E-rosetting. Inhibition activity of the peptide 6 in E-rosetting assay in the concentration range 0.0005-50 μM is shown in FIG. 3A. For comparison, inhibition activity of the control peptide is also shown. Peptide 6 showed inhibitory activity with IC₅₀ of 9.4±0.3 nM in the E-rosetting assay and 6.9 nM in OVCAR-T cell inhibition assay.

Peptide 6 reduces collagen-induced arthritis score. A preclinical animal model of CIA in DBA/1 mice was used to evaluate the ability of peptide 6 to suppress arthritis in a therapeutic protocol. Peptide P6 was administered via i.v. injection starting on day 22 after inducing arthritis and then five doses on alternate days. The disease progression was evaluated by visual appearance of the limbs in mice and was scored blindly. The mean arthritis score was decreased significantly in peptide 6-treated mice compared with control peptide-treated and untreated mice (FIG. 3B). Among the different doses of peptide 6 used for treatment, 1 mg/kg produced a significant reduction in arthritis score on days 40-46. There was a significant difference between the control peptide and peptide 6 at different concentrations as indicated by one-way ANOVA nonparametric analysis (p=0.0058). To determine the statistical significance and variability within the treatment groups, a Mann-Whitney test was performed using a 95% confidence interval. There was a significant difference between treatment groups with 1 and 0.25 mg/k (p=0.028). A Mann-Whitney test between control peptide and peptide 6 at 1 mg/kg indicated significant differences on days 40-46 (p=0.029). Because the mice did not show acute clinical signs of illness (i.e., anorexia, hunched posture, ruffled fur, and/or lethargy) during dosing or prior to the onset of observed clinical arthritis or any early mortality, these observations suggest minimal, if any, direct toxicity of the peptide (Gokhale et al. Chem. Biol. Drug Des. 2013, 82: 106-118).

Peptide 6 reduces histological features of collagen-induced arthritis. Histopathology of paws showed no abnormality in control animals, while arthritic mice with severe disease showed erosion of cartilage and bone resorption (FIG. 3Ci-iv). There was infiltration of neutrophils, mixed mononuclear inflammatory cells, and fibroblasts (FIG. 3Cii-iv). Animals treated with 0.5 mg/kg peptide 6 showed clear joint space with normal cartilage interface. Connective tissue surrounding joints was very minimally infiltrated with mixed inflammatory cells (FIG. 3Cv), suggesting that peptide 6 suppresses the progression of arthritis in the CIA model (Gokhale et al. Chem. Biol. Drug Des. 2013, 82: 106-118).

Peptide 6 reduces levels of circulating Collagen II antibodies. A role of autoreactive T cells and B cells is implicated in CIA. The autoimmune response to CII can be evaluated by measuring the quantity of the CII-specific antibody in the sera of the mice. To determine whether peptide 6 can inhibit the production of anti-CII Abs, serum samples were analyzed at the termination of the study in seven different groups of mice—control group without any treatment, arthritic, treatment group (three groups), vehicle control (PBS), and control peptide. Animals that were administered peptide 6 at 0.5 and 1 mg/kg showed a dose-dependent reduction in the anti-CII antibodies in sera. (FIG. 3D), suggesting that blocking interaction between CD2 and CD58/CD48 was able to inhibit humoral immune response in mice (Gokhale et al. Chem. Biol. Drug Des. 2013, 82: 106-118).

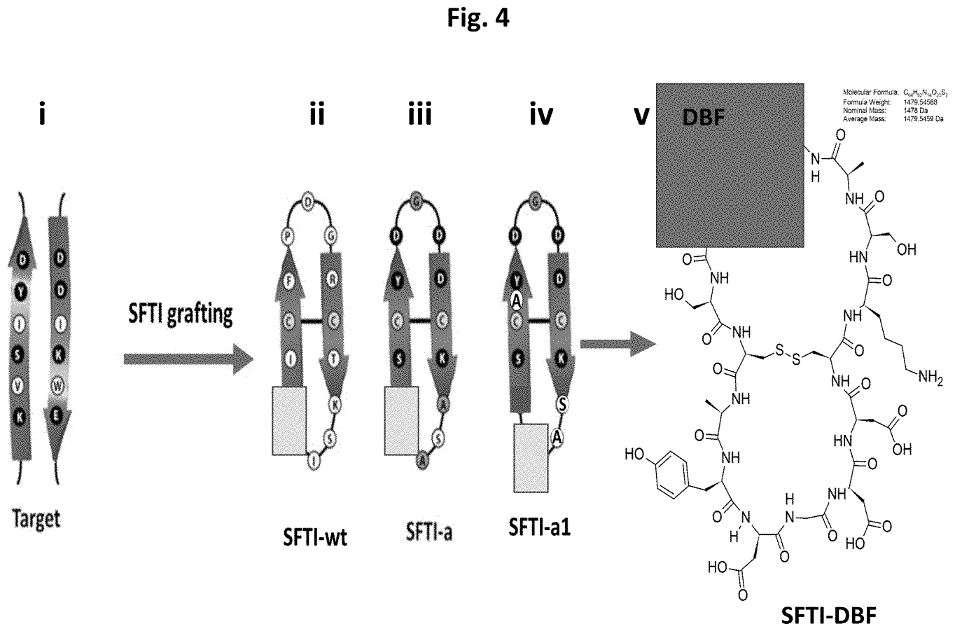

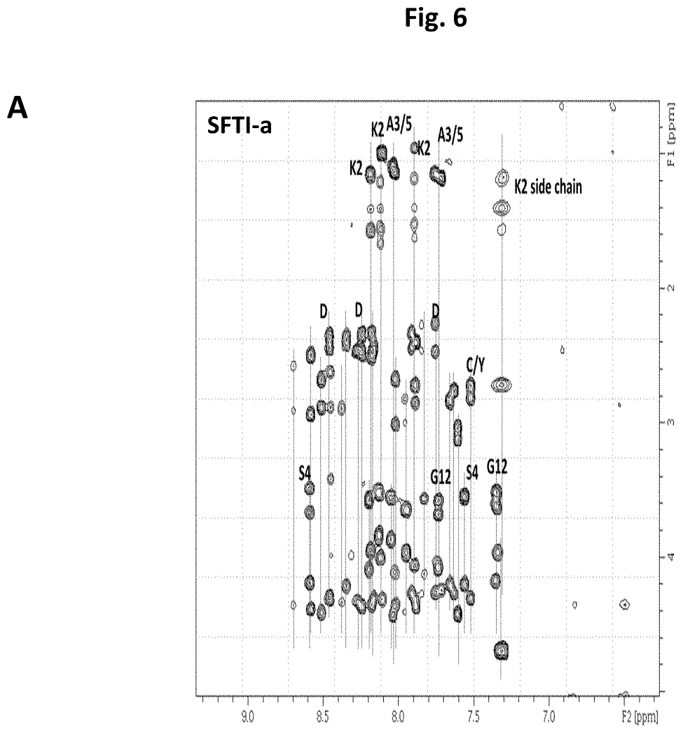

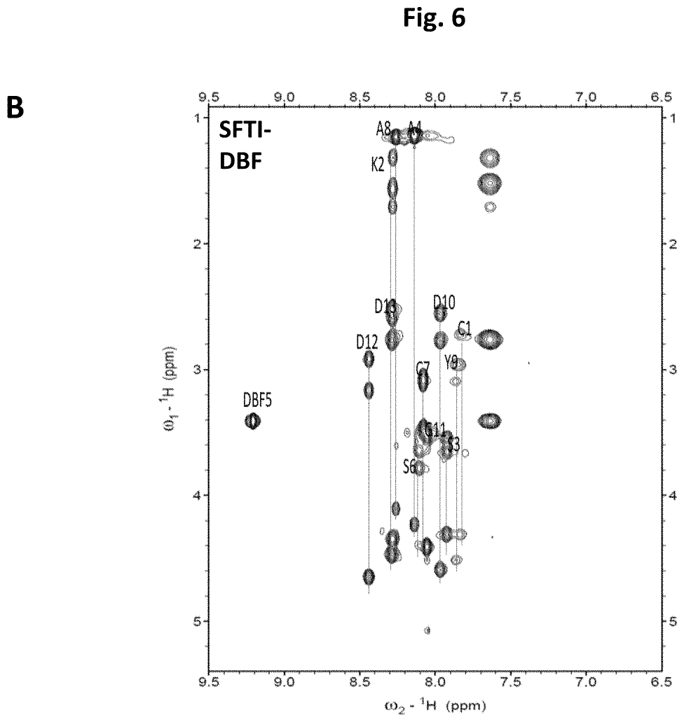

Design of CD2 peptidomimetic locked in beta conformation. CD2 peptides have β-strands in their structures placed nearly 5 Å apart, which is suitable for grafting CD2 adhesion epitope. In order to improve the stability of the peptides, we grafted the CD2 peptide epitope (FIG. 4i) onto sunflower trypsin inhibitor (SFTI) (FIG. 4ii) to create SFTI-a (FIG. 4iii) and SFTI-a1 (FIG. 4iv). SFTI-a1 is a version of SFTI-a that was modified using alanine scanning. In published studies, we showed that peptides grafted to SFTI are able to inhibit cell adhesion and suppress the immune response of T cells derived from mice with RA. However, peptides SFTI-a and SFTI-a1 both exhibited multiple conformers in solution as the native SFTI. Using two dimensional NMR, the inventors have shown that SFTI-a exhibits at least three possible conformations in solution as shown by multiple sets of NMR resonances for each amino acid residue in the peptide (FIG. 6A). By introducing the DBF moiety at a crucial position in the peptide (FIG. 4v), the inventors were able to constrain the peptide conformation in solution to one major conformation in solution as shown by 2D NMR in FIG. 6B. The designed peptides target cell surface proteins to modulate the signal generated by PPI; hence, the peptides do not have to cross the cellular membrane.

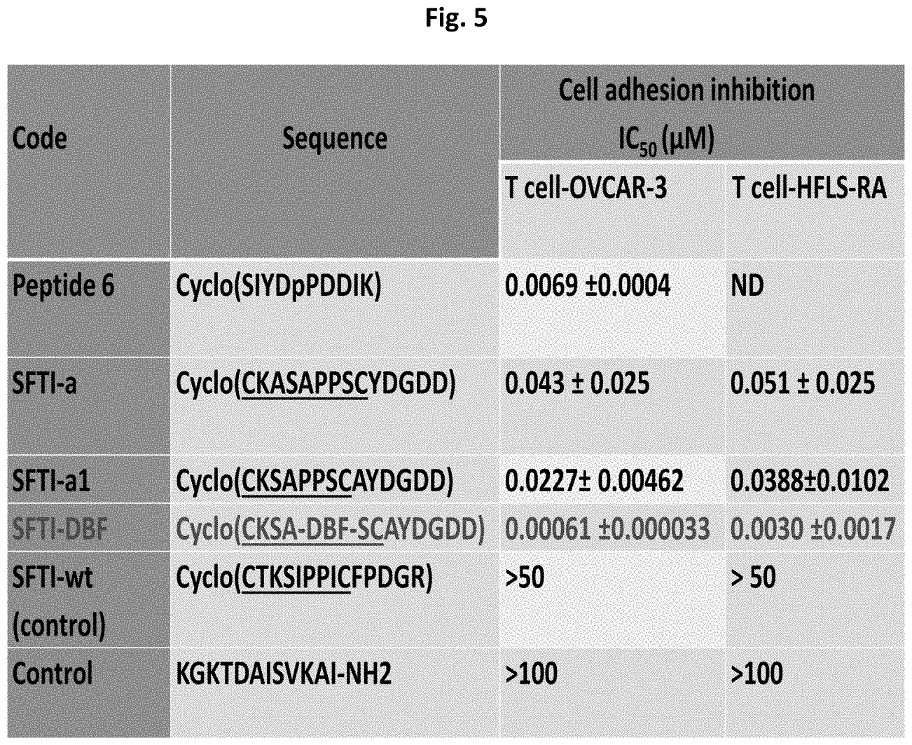

CD2 peptidomimetic locked in beta conformation is more potent than unlocked version. The ability of peptides to inhibit the CD2:CD58 protein-protein interaction was studied by evaluating the adhesion interaction between cells using model systems. T cells express the CD2 protein, and CD58 is expressed by many epithelial cells, as well as antigen-presenting cells. Many studies related to CD2 expression and CD2 signaling use Jurkat T cells. This cell line serves as a good model and has stable expression of CD2. Furthermore, in many drug discovery and mechanistic screening studies, model cell lines that express particular proteins are used. The model systems we used included Jurkat and ovarian cancer (OVACR-3) cells and Jurkat and human fibroblast-like synoviocyte-rheumatoid arthritis (HFLS-RA) cells. Since OVCAR-3 cells and HFLS-RA cells express CD58, adhesion interactions between CD2 and CD58 can be studied using these model systems. Peptides were incubated with adherent cells that expressed CD58, and Jurkat cells were added to evaluate the adhesion. Inhibition of adhesion was monitored by fluorescence of Jurkat cells loaded with 2′,7′-bis(2-carboxyethyl)-5-(and -6)-carboxyfluorescein acetoxymethyl ester (BCECF AM). A broad range of concentrations (0.0005 to 200 μM) was used in the first screen and based on the inhibitory activity of the peptides in that concentration range, data points for a dose-response study were chosen. The concentration-response curves for the various peptides were obtained in the range of 0.0005 to 10 μM. Among these peptides, SFTI-a1, an SFTI peptide grafted with a CD2 protein epitope, inhibits the cell adhesion between T cells that express CD2 and epithelial cells that express CD58 with an IC₅₀ value of 22 nM (FIG. 2vi). The new designed peptide SFTI-a1 is superior to previously designed SFTI-a (FIG. 2v) in terms of potency (IC₅₀ of cell adhesion 22 nM). Among the peptides the inventors designed, SFTI-DBF is the most potent for inhibiting PPI of CD2-CD58 in model systems using cellular assay (FIG. 5). The restriction of conformation in the peptide resulted in enhanced cell adhesion inhibition activity of the peptide in a cellular assay with an IC₅₀ value of 3 nM. Thus, the stable SFTI-a1-DBF (SFTI-DBF) peptide is the most potent peptide among the peptides the inventors designed (FIG. 5). The inventors believe that the potency of this peptide stems from a constrained structure that is restricted to one conformation.

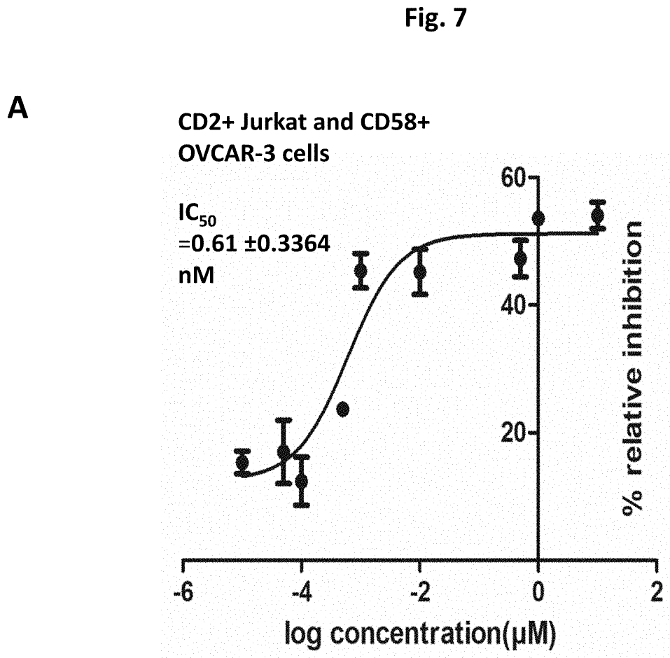

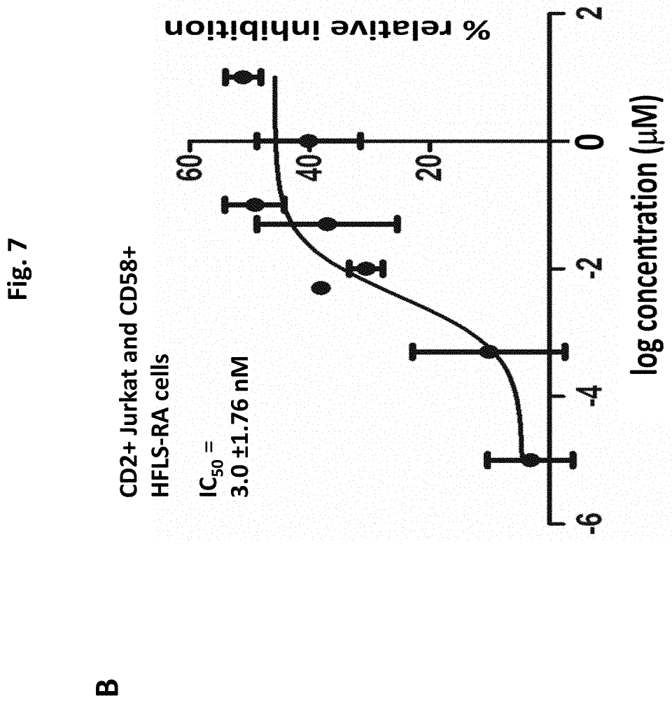

Cell adhesion was also evaluated in a well-established E-rosetting assay. SFTI-DBF inhibited adhesion between CD2+ Jurkat T cells and CD58+ OVCAR-3 cells with an IC₅₀ of 0.61±0.3364 nM (FIG. 7A). To evaluate the cell adhesion inhibitory activity of peptides from cells associated with autoimmune disease, we used HFLS-RA cells derived from RA patients. SFTI-DBF inhibition cell adhesion between CD2+ Jurkat T cells and CD58+ HFLS-RA cells with an IC₅₀ of 3±1.76 nM (FIG. 7B).

Conclusions. Peptide sequences from the cell adhesion/costimulatory molecule CD2 were grafted onto cyclic peptide frameworks. The designed peptides were evaluated for their cell adhesion inhibitory activity using model cell systems, as well as HFLS-RA cells derived from human RA. The peptides exhibited cell-cell adhesion inhibitory activity in the low nanomolar range and locking the SFTI-a1 peptide in beta conformation enhanced inhibitory activity. Such peptides that modulate the immune response by modulating protein-protein interaction of costimulatory molecules may be utilized as therapeutic agents for autoimmune diseases like RA.

The presently disclosed invention relates to therapeutics and methods of treating immune disorders, comprising the functional groups of a CD58-targeted peptide grafted onto a cyclic scaffold. According to further embodiments one or more of the functional groups of the peptide bind CD58. According to further embodiments the scaffold is chosen from the group of sunflower trypsin inhibitor (SFTI) and theta-defensins. According to further embodiments the targeted peptide is chosen from the group of:

Peptide 6
Cyclo(SIYDpPDDIK)
SFTI-a
Cyclo(CKASAPPSCYDGDD)
SFTI-a1
Cyclo(CKSAPPSCAYDGDD)
SFTI-DBF
Cyclo(CKSA-DBF-SCAYDGDD)
SFTI-wt (control)
Cyclo(CTKSIPPICFPDGR)
Control
KGKTDAISVKAI-NH2

Single letter code is used for amino acid representation. Capital letters refer to L-amino acid, small letter refers to D-amino acid (P is L-proline, p is D-proline). Cyclo indicates a cyclic compound. DBF is dibenzofuran. Disulfide bond is indicated by underline. According to further embodiments the immune disease is chosen from the group of rheumatoid arthritis, psoriatic arthritis, plaque psoriasis, ankylosing spondylitis, Crohn's disease, and ulcerative colitis. According to further embodiments cells associated with the immune disease are CD2-positive. According to further embodiments cells associated with the immune disease are CD58-positive. According to further embodiments, the method is used in combination with an effective amount of one or more immune therapy agents from the group of etanercept, adalimumab, infliximab, certolizumab pegol, and golimumab. According to further embodiments the administration route is oral, intravenous, or inhalation. According to further embodiments the peptide is administered with pharmaceutically acceptable excipients. According to further embodiments the peptide sequence freely changes conformations or is locked into beta conformation. According to further embodiments the peptide sequence is locked into beta conformation through incorporation of 1,4 substituted triazole or substitution of Pro-Pro with dibenzofuran.

The disclosed invention also relates to the peptides:

SFTI-a1
Cyclo(CKSAPPSCAYDGDD),
and
SFTI-DBF
Cyclo(CKSA-DBF-SCAYDGDD),

and methods of treatment using the peptides. Single letter code is used for amino acid representation. Capital letters refer to L-amino acid (P is L-proline). DBF is dibenzofuran. Disulfide bond is indicated by underline.

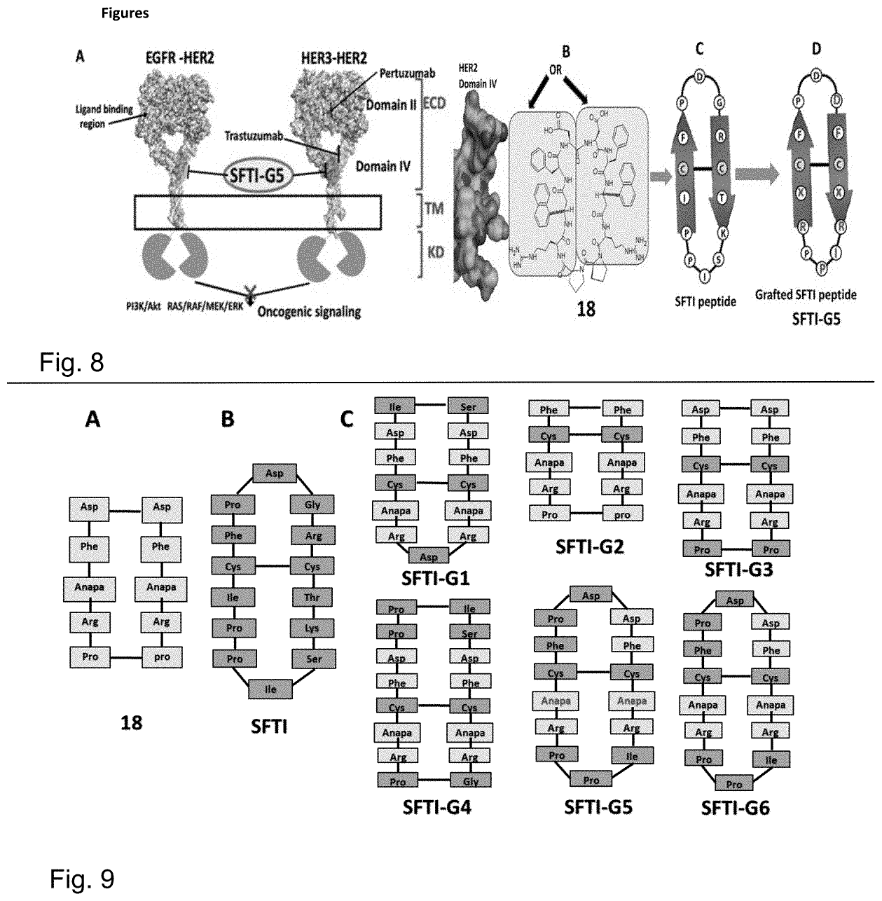

The five-year relative survival rate for lung cancer patients is known to be around 18%, only a slight increase over what it was more than a decade ago, indicating that improvements in lung cancer therapy have been slow. Among the lung cancers, about 85% are histologically classified as non-small-cell lung cancer (NSCLC), a leading cause of cancer deaths worldwide. Genotype-driven therapy or targeted therapy approaches are promising treatments for lung cancer. Epidermal growth factor receptors (EGFRs) are known to play a crucial role in lung cancer. The receptor family consists of four members: HER1 or EGFR and HER2-4. These proteins have an extracellular domain (ECD), a transmembrane helix, a cytoplasmic kinase domain, and a regulatory region. Ligand binding to EGFR or HER3 ECDs triggers a change in the conformation of the proteins, leading to their heterodimerization (FIG. 8) and, ultimately, to cell signaling. Spontaneous dimerization of HER2 occurs via gene amplification or kinase activation by EGFR or HER3. In HER2-related NSCLC, three mechanisms of HER2 activation have been described: a) HER2 protein overexpression, b) HER2 gene amplification, and c) HER2 gene mutations. These mechanisms also seem to have a role in the ability to develop resistance to targeted antibodies and tyrosine kinase inhibitors (TKIs). HER2 overexpression has been reported at different frequencies in NSCLC patients with extremely wide ranges. HER2 overexpression is detected by immunohistochemistry in 13%-20% of NSCLC samples, and strong expression is found in only 2%-4%. HER2 amplification is considered an alternative mechanism for the development of resistance to EGFR-targeted TKI therapy. HER2 mutations and amplification are seen in 3% of lung cancers. Thus, targeting HER2 and EGFR and their protein-protein interaction (PPI) or dimerization is important in NSCLC. The co-expression of EGFR and HER2 in NSCLC patients was associated with a significantly shortened overall survival rate compared with that of cancer patients whose tumors had high levels of EGFR or HER2 alone.

EGFR has been targeted with three generations of TKIs, and drug resistance has become a major issue. EGFR-mutant cells can become resistant by acquiring the T790M mutation. However, targeted therapies currently approved for NSCLC show promise. Furthermore, mutant EGFRs, especially the L858R/T790M variant, have a propensity to heterodimerize with HER2. Thus, HER2 plays a significant role in mediating the sensitivity of EGFR-mutant lung tumors to anti-EGFR therapy. Taken together, these studies indicate that receptor signaling of EGFR, HER2, and HER3 is interdependent and emphasize the importance of inhibiting more than one HER family receptor for blockage of the signaling network of HER family receptors. Thus, inhibition of dimerization of EGFR:HER2 and HER2:HER3 signaling is necessary to achieve maximal anti-tumor responses in NSCLC therapy. Other approaches, such as immune checkpoint inhibitors, have been approved as immunotherapy for NSCLC but are limited to their expression in tissues. The use of monoclonal antibodies or a combination of antibodies with traditional chemotherapeutic agents or with kinase inhibitors has been widely seen in recent years. However, the antibodies that have been targeted to HER2 and EGFR proteins for the treatment of cancer have limitations in terms of stability, immunogenicity, size, cardiotoxicity, and cost. Thus, there is a need for the development of novel molecules that are non-immunogenic and devoid of cardiotoxicity.

Extracellular domains of EGFRs homo- and heterodimerize by interacting with domain II and domain IV protein-protein surfaces (FIG. 8). Crystal structures of EGFR dimers suggest that domain IV interactions in the dimer are dominated by hydrophobic residues such as Leu, Trp, and Tyr. Our interest is in targeting these with grafted peptides to modulate the cell signaling by inhibiting protein-protein interaction (PPI) “hotspots” in dimerization. For peptides/peptidomimetics, stability is a major area of concern when considering therapeutic agents. Peptides are susceptible to enzymatic and chemical degradation. To improve the stability of peptides, different approaches have been considered, such as N- and C-terminal modification, cyclization, side-chain modification, and chirality modification. The novel use of cyclotide-like multicyclic frameworks to graft an active peptide sequence is a more advanced method of stability improvement. Grafting of peptides onto cysteine-rich scaffolds is in high demand and has been successful in many studies.

Sunflower trypsin inhibitor (SFTI-1), a cyclic peptide comprised of 14 amino acids found mainly in sunflower seeds. The structure consists of a cyclic backbone, and is stabilized by a disulfide bridge. Due to its small size, extensive hydrogen-bonding network, and compact rigidity, it is one of the most widely employed molecular scaffolds for drug discovery. In our previous work, we have shown that a peptidomimetic compound 18 binds specifically to ECD of HER2 and inhibits ECD heterodimerization of EGFRs, thereby preventing the downstream signaling by EGFR proteins (Table 1). Although the designed peptide had potent in vitro and in vivo activity toward NSCLC, like most other peptides, it has limitations to its in vivo stability. To overcome these, we have designed bicyclic peptides grafted onto a plant peptide, SFTI-1, to inhibit EGFR dimerization as well as improve stability (FIG. 8A). Among the designed peptides, SFTI-G5, an SFTI peptide grafted with a previously developed compound in our laboratory, exhibited antiproliferative activity with an IC₅₀ value of 0.073 μM in HER2-overexpressing lung cancer cell line Calu-3 (FIG. 8D) and was highly specific for HER2+ cancer cell lines. It also inhibited the dimerization of EGFR:HER2 and HER2:HER3 protein-protein interactions, thereby inhibiting the phosphorylation of HER2 via the Akt pathway. In addition, our results suggest that compound SFTI-G5 could suppress tumor growth in an animal model based on the in vivo study in mice.

Results: Design and synthesis of cyclic grafted peptides: In our previous studies, we have reported that a peptidomimetic, compound 18, inhibited protein-protein interactions between EGFR-HER2 and HER2-HER3 in NSCLC cell lines and exhibited anti-tumor efficacy in an in vivo mouse model of breast cancer. With the aim of improving the stability of the peptidomimetics while maintaining or enhancing their potency, the peptidomimetic with the most potent anti-tumor activity to inhibit EGFR:HER2 dimerization (compound 18) was grafted onto the SFTI-1 framework (Table 1) to generate different grafted peptidomimetics. We used the disulfide-stabilized, antiparallel β-strand conformation of SFTI-1 (PDB ID: 1JBL) for our design. Retaining the disulfide bond of the SFTI framework, we replaced the amino acids of SFTI with Arg-Anapa-Phe-Asp. L-Pro-D-Pro in compound 18 was used to stabilize the structure and, hence, L-Pro-D-Pro from compound 18 was not grafted onto the SFTI framework. We compared the 3D structure of 18 and SFTI-1 by overlapping the backbone atoms. Since the number of amino acids in the two peptides are different and the binding of particular amino acid side chain function group to HER2 protein is not well established in 18, we had to design several possible grafted peptides.

In addition, SFTI-1 has three proline amino acids, and some amino acids such as Asp, Phe, and Arg are also present in compound 18 and the SFTI framework (FIGS. 8 and 9). Thus, grafting was done with different possibilities without removing disulfide bonds in the SFTI framework. For example, in SFTI-G1, Ile-Ser of the loop region, as well as Asp amino acids and the disulfide bond from SFTI, were retained, and Pro amino acids were deleted (FIG. 9). Arg-Anap-Phe-Asp from 18 was grafted. In SFTI-G2, only a disulfide bridge was retained, and the entire SFTI framework was replaced with Arg-Anapa-Phe-Pro from compound 18 to reduce the total number of amino acids in the SFTI framework. In SFTI-G3, Arg-Anapa-Phe-Asp-Pro from 18 and the disulfide bond from the SFTI framework were used. SFTI-G4 was designed keeping 8 amino acids of the SFTI framework and introducing Arg-Anapa-Phe-Asp from 18. In SFTI-G5 and SFTI-G6, the total number of amino acids (14) in the SFTI framework was maintained, including the Ile-Pro-Pro loop region, and the remaining amino acids were replaced by Arg-Anapa-Phe-Asp (FIG. 8C). To assess the role of chirality of a β-amino acid, namely, 3-amino-3-(1-naphthyl propionic acid) (Anapa), on biological activity, we synthesized compounds of both S-Anapa and R-Anapa.

Peptide synthesis and characterization. The designed grafted peptides were synthesized and characterized by Fmoc solid-phase peptide synthesis as reported earlier. The sequences of the grafted cyclic peptides are shown in Table 1. The peptides were purified by preparative RP-HPLC. The purity of the grafted peptides was analyzed by analytical HPLC and high-resolution mass spectrometry (HR-MS).

Antiproliferative activity of grafted cyclic peptides. Antiproliferative activity of the designed grafted peptides was evaluated in different HER2-overexpressing cancer cell lines as well as HER2 negative cancer cell lines. In a Cell Titer-Glo assay that indicates the viability of cells, it was shown that one of the grafted peptides, SFTI-G5 exhibited, an IC₅₀ value of 0.280 μM in HER2-overexpressing breast cancer cell line BT-474 and 0.073 μM in lung cancer Calu-3 cell line. In the A549 cell line, which is an EGFR wild-type and KRAS mutated NSCLC cell line, the activity was found to be 0.369 μM. Furthermore, in the MCF-7 cell line, which is a HER2-negative cell line, the IC₅₀ was found to be >25 μM (Table 1). The dose-response curves for SFTI-G5 in different cell lines are depicted in. In the case of non-cancerous breast epithelial cell line MCF-10A, SFTI-G5 exhibited antiproliferative activity with an IC₅₀ value of >50 μM. Thus, we can conclude that the SFTI-G5 has high specificity toward HER2-overexpressing cancer cell lines. The activity of the designed grafted peptides was reduced slightly in all the HER2+ cell lines compared to the parent peptidomimetic 18 (Table 1). This loss of activity may be due to altering positions of the vital amino acids in the SFTI framework, which might be essential for interactions with the HER2 protein hot-spot region. We also evaluated the effect of the chirality of β-amino acid in SFTI-G5 on the antiproliferative activity. By altering the chirality of Anapa (from S-Anapa to R-Anapa) (SFTI-G6) (Table 1), there was a tenfold decrease in antiproliferative activity. This indicates the importance of the chirality of the Anapa group in the grafted peptide. As SFTI-G5 exhibited the highest antiproliferative activity in HER2-overexpressing cancer cell lines among the grafted peptides designed, further studies related to the molecular mechanism and dimerization inhibition as well as in vivo studies were carried out on SFTI-G5.

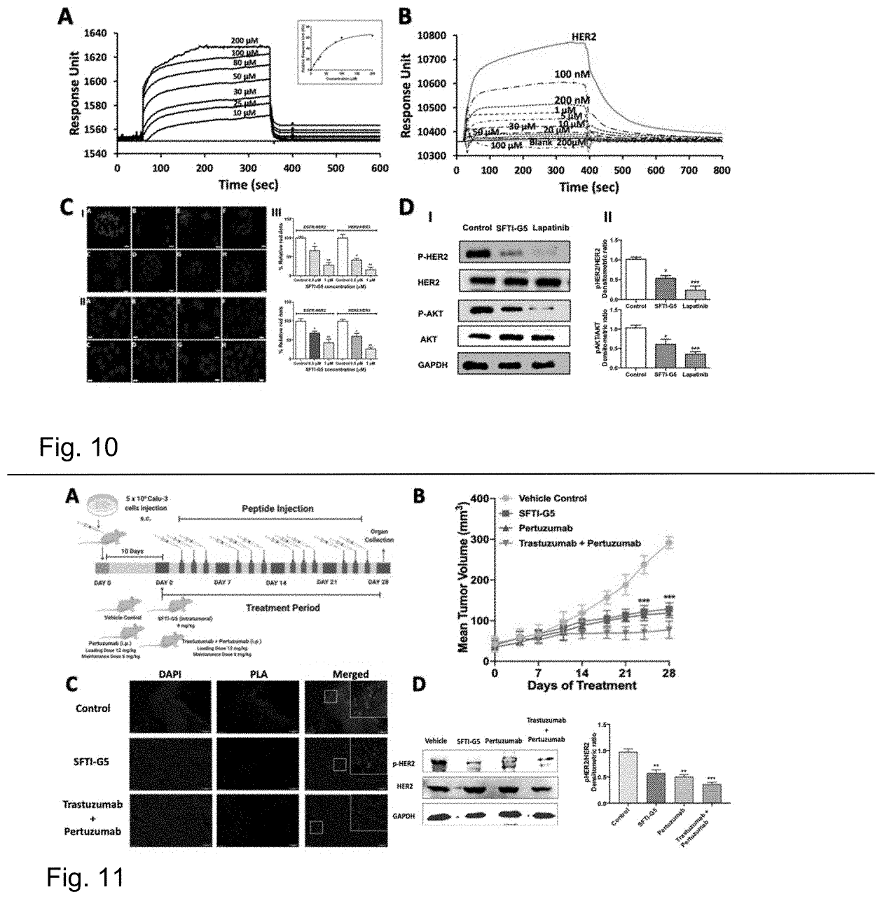

Binding of SFTI-G5 to HER2 ECD. Compound 18 was shown to bind to the HER2 ECD and inhibit the PPI of EGFRs. Since SFTI-G5 was designed from compound 18, we assume that SFTI-G5 binds to the ECD of HER2 and inhibits the PPI of EGFRs. To confirm that the grafted peptide, SFTI-G5, binds to the ECD of HER2, we performed peptide to protein binding studies using surface plasmon resonance (SPR). We performed an SPR study by immobilizing HER2 ECD protein (domains I-IV, 620 amino acids) and its domain IV of ECD onto the CM5 sensor chip separately, and SFTI-G5 was injected over the immobilized chips at different concentrations. The SPR sensorgrams showed concentration-dependent binding of SFTI-G5 to HER2 ECD (FIG. 10A) as well as to domain IV. Binding affinities were obtained by global fitting analysis of the titration curve to the 1:1 Langmuir interaction model, and the K_{d} value was calculated. The binding affinities (K_{d} values) of SFTI-G5 for HER2 ECD and domain IV of HER2 protein were found to be 4.87×10⁻⁷ M and 3.46×10⁻⁷ M, respectively. When a control compound (Table 1) was used as an analyte, no SPR response was recorded, indicating specific binding of SFTI-G5 to HER2 ECD.

Further, to confirm that SFTI-G5 inhibits the heterodimerization of EGFR-HER2 by binding to HER2, we performed a competitive binding study with SPR. Here, EGFR protein was immobilized on CM5 chip, and varying amounts of SFTI-G5 (200-1 μM) were injected with a fixed concentration of HER2 protein (200 nM). When HER2 protein (200 nM) was injected alone, a response unit of 350 was recorded, which suggested protein-protein interactions of EGFR and HER2. As the concentration of SFTI-G5 increased in the presence of HER2 protein, we observed a decrease in the response unit (FIG. 10B), suggesting that our compound competitively binds to the HER2 protein and prevents the dimerization of HER2 with EGFR. Hence, we can confirm that SFTI-G5 binds specifically to HER2 protein at the dimerization site of domain IV of HER2 and inhibits the dimerization of EGFR and HER2 protein.

Inhibition of EGFRs dimerization by proximity ligation assay (PLA). Our aim is to inhibit the PPI of EGFR and, thus, modulate the cell signaling for cell growth. SFTI-G5 was designed to inhibit the EGFR heterodimers. To show that SFTI-G5 inhibits EGFR heterodimerization, we performed (PLA) in Calu-3 and A549 NSCLC cell lines. PLA is an established technique to quantify protein-protein interactions in cells. Calu-3 cells were incubated with different concentrations of SFTI-G5, and PLA assay was carried out using different antibodies (EGFR, HER2, and HER3). Cells without SFTI-G5 treatment were considered a positive control and cells not treated with the primary antibody but with a secondary antibody were regarded as another control. Pertuzumab, an antibody that is known to bind to HER2 ECD domain II and inhibit the dimerization of EGFRs, was used as a positive control. In the absence of the SFTI-G5, we observed a high number of red fluorescence dots in the cells, indicating EGFR:HER2 dimerization in Calu-3 cells (FIG. 10CI). Here, each red dot represents the dimerization of EGFR:HER2 and HER2:HER3 proteins. In the presence of SFTI-G5 at 0.5 μM and 1 μM concentrations, the number of red dots was significantly lower than in the positive control (FIG. 10CI). Our results indicate that SFTI-G5 inhibits heterodimerization in a concentration-dependent manner. We also found similar results when we probed for HER2:EGFR dimers with PLA probes (FIG. 10CI). Similar results were observed when PLA assay was performed on A549 cells with and without SFTI-G5 (FIG. 10CII). Quantification of red fluorescence from PLA suggested that SFTI-G5 significantly inhibited the dimerization of HER2 compared to control in both cell lines (p<0.05 for 0.5 μM and p<0.01 for 1 μM SFTI-G5 concentration) (FIG. 10CIII). These results suggest that SFTI-G5 is a dual inhibitor of dimerization of EGFR heterodimers, EGFR:HER2 and HER2:HER3. Also, we compared SFTI-G5 against Pertuzumab, a HER2 dimerization inhibitor, for the inhibition of HER2 dimerization which showed similar result to Pertuzumab for inhibiting dimerization of HER2.

SFTI-G5 inhibits phosphorylation of HER2 kinase and modulates downstream signals. To confirm the inhibition of PPI of ECD of EGFRs by SFTI-G5 and its effects on intracellular signaling, Western blot analysis was performed on NSCLC cells. A549 that overexpress HER2 protein was incubated with SFTI-G5 and, after extraction of HER2 protein, the amount of phosphorylated protein was determined by Western blot (FIG. 10D). The phosphorylation of HER2 was measured by the p-HER2 monoclonal antibody. Quantitative analysis of Western blot indicated that SFTI-G5 significantly decreases the phosphorylation of HER2 kinase compared to control (without peptide treatment). Lapatinib, an EGFR and HER2 kinase dual inhibitor were used as a positive control. The results suggest that the binding of SFTI-G5 to the ECD inhibits the phosphorylation of the intracellular kinase domain of HER2. However, there was no significant change in total HER2 protein levels (FIG. 10D). To understand whether SFTI-G5 inhibits any down-stream signaling molecules, we further evaluated the phosphorylation of Akt. As indicated in FIG. 10DI, SFTI-G5 was able to inhibit the phosphorylation of Akt. Quantification of Western blot suggested that SFTI-G5 significantly inhibited the phosphorylation of Akt compared to control (FIG. 10DII).

Molecular Docking: To model, the interactions of SFTI-G5 with HER2 protein ECD, a docking method was used. Docking of the grafted peptide to SFTI-G5 was performed using AUTODOCK. A three-dimensional structure of SFTI-G5 was generated using InsightII (BIOVIA, San Diego, Calif., USA) molecular modeling software by the energy minimization method. The grafted peptide SFTI-G5 was docked around the region of domain IV of HER2 ECD. The low-energy structures that had energy values within 2 kcal/mol of the lowest energy docked structures were analyzed as possible binding modes. Ten structures within 2 kcal/mol energy were chosen as possible binding modes for SFTI-G5. The lowest energy docked structure had a docking energy of −9.99 kcal/mol. It was found that all the structures were docked at the C-terminal portion of domain IV of HER2. The lowest energy structures with docked conformation are shown herein. The peptide was found to form hydrophobic interactions with HER2 domain IV. The β-naphthyl group of SFTI-G5 participates in a hydrophobic interaction with Pro584 of HER2 domain IV. Hydrogen bonding was observed in the lowest energy docked structure between two arginines of peptide and Asp585 and Tyr588, respectively. Also, another hydrogen bonding was observed with aspartic acid of peptide and Lys593 of HER2. These docking studies provided a model for binding of SFTI-G5 to HER2 protein domain IV and suggested important amino acid interactions responsible for binding and binding sites of the protein.

Stability of the secondary structure of SFTI-G5. Due to its cyclic nature and stabilized disulfide bridge, the parent SFTI-1 molecule typically exhibits higher thermal stability than natural peptides, which are prone to conformational change with increase in temperature. We wanted to confirm whether, after the grafting, SFTI-G5 retains thermal stability. Circular dichroism spectroscopy was used to evaluate the thermal stability of grafted peptide SFTI-G5. There were no significant changes in the CD spectra of SFTI-G5 recorded at different temperatures (25-75° C.), which suggested that the overall secondary structure of the peptide remained the same at different temperatures. To confirm further, a mass spectrum of SFTI-G5 was obtained from the solution used to record the CD spectrum at different temperatures. Freeze-dried samples of different temperatures were analyzed via MALDI-TOF mass spectrometry. Samples of SFTI-G5 from 25 to 75° C. showed intact molecular ion for the compound, suggesting that SFTI-G5 was stable at 75° C. Thus, the CD and mass spectrometry results confirm that SFTI-G5 is stable against thermal denaturation.

To further evaluate the stability of the disulfide bond, the CD spectrum of SFTI-G5 was examined in the presence and absence of dithiothreitol (DTT). There was a sharp change in CD spectrum of SFTI-G5 with the addition of DTT, suggesting that a reduction in the disulfide bond changes the conformation of the peptide. The reduction was further confirmed by a shift in the molecular ion of SFTI-G5 upon the addition of DTT (m/z 1839 to 1841 after the addition of DTT,

In vivo studies. To obtain the proof-of-concept of SFTI-G5 dimerization inhibition and effect on NSCLC tumor growth in an animal model, we carried out in vivo studies on two NSCLC models.

Effect of SFTI-G5 on tumor growth in a xenograft model of lung cancer. To evaluate whether the designed grafted peptide SFTI-G5 could reduce the progression of lung cancer tumor growth in aa animal model, mice with xenograft tumors were treated with SFTI-G5 at 8 mg/kg thrice a week via intratumor injection just below the tumor. HER2 dimerization inhibitors such as pertuzumab and trastuzumab and pertuzumab combinations were also administered as control groups (FIG. 11A). Based on our hypothesis, SFTI-G5 is expected to inhibit HER2 heterodimerization in vivo, thus leading to delayed tumor growth progression. During the course of the experiment, the tumor size in the control xenograft group without any treatment continued to increase, reaching a diameter of ˜9 mm in 28 days (FIG. 11B).

On the other hand, SFTI-G5 showed a delay in tumor growth that was significant compared to control with p<0.001 (FIG. 11B). Based on the results of the two-tailed student t-test for statistical significance, treatment with pertuzumab also significantly reduced tumor growth progression compared to vehicle control and SFTI-G5. The combination treatment of trastuzumab and pertuzumab showed enhanced antitumor activity compared to both monotherapies. Further, the dosing schedule of SFTI-G5 as monotherapy was well-tolerated and had no significant effects on body weight or clinical observations (not shown).

To evaluate the phosphorylation of HER2 protein, Western blot analysis was performed on frozen tumor samples. Results from Western blot analysis of the tumor samples showed significant inhibition of HER2 phosphorylation by SFTI-G5 compared to vehicle treatment (FIG. 11D). Thus, SFTI-G5 binds to the ECD and inhibits the phosphorylation of HER2.

To confirm whether SFTI-G5 inhibited the heterodimerization, fixed tumor sections from SFTI-G5-treated, trastuzumab- and pertuzumab-treated, and vehicle control groups were incubated with primary and secondary antibodies of HER2 and EGFR, and PLA assay was carried out. Samples from tumors treated with SFTI-G5 exhibited markedly diminished signals from dimerization as indicated by a relative decrease in red fluorescence compared to the control tumor sample, indicating a decrease in the heterodimerization of EGFR:HER2 in vivo (FIG. 11C). The results suggest that inhibition of dimerization inhibits the signaling for cell growth and correlates with the reduction of tumor growth progression, as observed in the antitumor study.

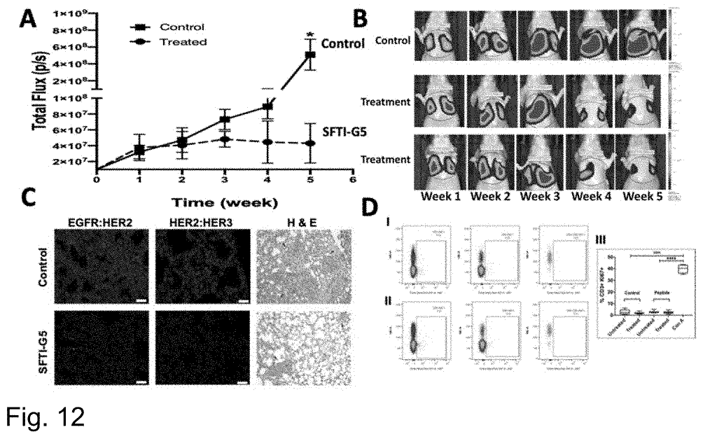

Effect of SFTI-G5 on tumor growth in an experimental metastasis model of lung cancer. In the experimental metastasis model, the tumor can grow in particular organs following intravascular injection. Before injecting to the animals, the Bioware® Brite A549 Red-FLuc luciferase transfected cells (PerkinElmer) were first evaluated in vitro for their bioluminescence efficiency. Once bioluminescence efficiency was confirmed, the cells were injected into the tail vein of mice. The reason for selecting A549 cells is that these are derived from NSCLC and are known to have KRAS mutation and EGFR wild-type. Mice were monitored for one week prior to i.p. injection with luciferin and bioluminescence was measured for each animal by imaging under anesthesia using an IVIS imaging system (Caliper Life Sciences) instrument. Using whole-body bioluminescence imaging, the growth of the tumor was monitored once a week. After two weeks of injection of cells, mice with lung tumors in were divided into two groups and injected intravenously with either vehicle or SFTI-G5 (6 mg/kg) in 1004 prepared in PBS twice a week. This dosage was based on our previously reported work on HER2+ breast cancer using peptidomimetics. All mice were imaged weekly with bioluminescence imaging. It has been reported that the photon flux from the tumor is directly proportional to the number of light-emitting cells that express luciferase, and that the signal can be measured to monitor tumor growth and development. As shown in FIG. 12A, after two weeks, the rate of tumor growth, indicated by total flux (p/s), decreased in mice treated with SFTI-G5 (6 mg/kg) compared with mice injected with vehicle. There was a significant difference in the tumor growth rate between control and treated groups (FIG. 12AB). Statistical analysis was done using the two-tailed student t-test. Mice were sacrificed on day 35, and excised lungs and other organs were individually analyzed.

To gain further understanding of the therapeutic effects of treatments on lung tumor, hematoxylin & eosin (H&E)-stained cross-sections of the lungs with tumors were studied. H&E staining of histological sections of the lungs from mice without treatment (vehicle control) showed the presence of enlarged, hyperchromatic nuclei and abundant eosinophilic cytoplasm (FIG. 12C), while in the SFTI-G5-treated group there were less.

Inhibition of EGFR dimerization by SFTI-G5. Tissue sections of lung tumor samples were further evaluated by proximity ligation assay for EGFR dimerization inhibition by SFTI-G5 in vivo. Tissue sections from the vehicle control group showed high red fluorescence, indicating EGFR:HER2 and HER2:HER3 dimerization. Sections of tissues that were treated with SFTI-G5 showed a reduction in red fluorescence, indicating inhibition of EGFR:HER2 and HER2:HER3 dimerization (FIG. 12C). Thus, SFTI-G5 inhibits both dimers in the in vivo NSCLC model. The results suggest that inhibition of the ECD of HER2 inhibits signaling for cell growth and, hence, reduction in tumor growth in the animal model of cancer.

Tissue sections of lung tumor samples were also evaluated for HER2 expression using FITC-labeled HER2 antibody. Tumor sections show the expression of HER2 in sections injected with A549 cells, as well as in those from the control group, which were not injected with A549 cells.

SFTI-G5 is not immunogenic in mice. Molecules that are not endogenous to the body can trigger cellular responses when administered. Although our previous studies suggest that peptides/peptidomimetics are non-immunogenic, grafting the peptide onto a plant-based peptide may induce immunogenicity. Hence, to investigate whether the grafted peptide SFTI-G5 is immunogenic and initiates any immune response after administration, we performed a flow cytometry-based T-cell proliferation assay in a mouse model. We evaluated the Ki-67 expressions in T cells of both control and the SFTI-G5 primed group using different T-cell markers (CD3, CD4, and CD8). Ki-67 is a proliferation marker of nuclear protein and has been used to measure specific T-cell responses. Here, the BALB/c mice were primed with SFTI-G5 at a dose of 6 mg/kg, followed by a booster dose on the 10th day. The animals were sacrificed on the 13th day, and splenocytes were harvested and cultured in vitro from both control and primed groups, either in the presence or absence of the peptide. Concanavalin A (5 μg/mL), an antigen-independent mitogen known to induce T-cell proliferation, was used as a positive control. After 48-h incubation, the cells were stained with surface markers as well as an intracellular staining marker, Ki-67. Flow cytometry data suggest that the expression of Ki-67 in CD3+ subsets (CD4+, CD8+, and CD 4+CD8+) in the SFTI-G5-treated group did not show any significant immune response at 5 μM compared to control and concanavalin A (p<0.0001) (FIG. 12D).

Discussion: EGFR and HER2 proteins are the major markers for NSCLC in addition to KRAS and ALK. EGFR resistance is one of the major problems with a mutation in the kinase domain. Our research interest is to target the dimerization of ECDs of EGFRs in order to avoid EGFR/HER2 kinase mutation. Although ECD mutation was observed in few cases, mutation of domain IV of EGFR and HER2 has not been detected thus far. Blocking HER2-mediated signaling by inhibiting PPIs that HER2 makes with EGFRs has potential therapeutic value. Inhibition of protein-protein interaction of EGFR:HER2 and HER2:HER3 dimerization prevents the downstream signaling pathways (AKT and MAPK) and affects cell proliferation, survival, and tumor growth. Monoclonal antibodies specifically directed against the ECD of HER2 have been shown to be selective inhibitors of the proliferation of HER2-overexpressing cancer cells. Trastuzumab binds to domain IV of HER2 and inhibits cell signaling for proliferation selectively in cancer cells. Previously, we have designed some peptidomimetics in our laboratory that have shown that dimerization of EGFR can be inhibited by peptidomimetics by targeting domain IV of HER2 and inhibiting the downstream signaling. In this project, our approach was to increase the stability of active peptides by grafting onto the SFTI framework, which is known to be stable at a higher temperature and prevents enzymatic/chemical degradation.

The present study describes an SFTI-grafted peptidomimetic, SFTI-G5, that targets the HER2 ECD, in particular, domain IV, to inhibit the interaction between HER2 and other EGFRs. We have used SFTI-1 for grafting an active peptidomimetic, compound 18, a lead compound from previous work, and have designed several analogs of the grafted multicyclic peptide. Out of several analogs of grafted peptides, one peptide, SFTI-G5, exhibited antiproliferative activity in HER2-overexpressing cell lines with nanomolar activity. In lung cancer cell line Calu-3, the antiproliferation activity was found to be 0.073 μM, while in A549 cells, it was 0.369 μM. Using SPR and PLA assays, we have shown that SFTI-G5 binds specifically to HER2 ECD domain IV and inhibits HER2:HER3 dimerization (FIG. 10). Thus, the designed compounds inhibit protein-protein interactions of HER2 and HER3 ECDs. Moreover, the compound inhibited heterodimerization of HER2 with other EGFRs and, thereby, phosphorylation of HER2, which is evident from the immunoblot analysis on Calu-3 cells (FIG. 10). SFTI-G5 showed high stability against thermal degradation and was stable up to 75° C. in solution.

In the experimental metastasis model of lung cancer, SFTI-G5 clearly exhibited a significant reduction in tumor growth compared to the negative control. In this method, A549 cells, which have EGFR/HER2 overexpression and KRAS mutation, were used via tail vein injection and lung cancer was successfully developed in nude mice. An in vivo study suggested that the treatment group significantly reduces tumor growth compared to the control group (FIG. 12). Further, PLA carried out on tumor sections suggested that SFTI-G5 inhibits the EGFR:HER2 as well as HER2:HER3 heterodimerizations under in vivo conditions. Taken together, these studies suggest that inhibition of HER2 heterodimerization with other EGFRs by SFTI-G5 hinders intracellular trans-phosphorylation, leading to controlled cell proliferation and tumor growth. In addition, in the immunogenicity study in the mouse model, we found that SFTI-G5 is non-immunogenic to animals even at high concentrations.

Conclusions. We successfully synthesized a series of grafted cyclic peptides by grafting potent peptidomimetics onto the SFTI-1 framework without altering the disulfide bridge. Among these grafted peptides, SFTI-G5 was found to have higher potency than others, as confirmed from the antiproliferative assay. Further, SPR results showed that SFTI-G5 binds to the ECD of HER2 protein, specifically Domain IV of HER2 protein. PLA and Western blot results showed that the grafted cyclic peptide inhibits the dimerization of EGFRs and ultimately reduces the downstream signaling. In vivo studies suggested that SFTI-G5 reduces tumor growth significantly compared to control. Stability studies indicated that the compound is retains its secondary structure even at high temperatures.

Pharmaceutical Compositions

The methods described herein can also include the administrations of pharmaceutically acceptable compositions that include the therapeutic, or a pharmaceutically acceptable salt, solvate, or prodrug thereof. When employed as pharmaceuticals, any of the present compounds can be administered in the form of pharmaceutical compositions. These compositions can be prepared in a manner well known in the pharmaceutical art, and can be administered by a variety of routes, depending upon whether local or systemic treatment is desired and upon the area to be treated. Administration may be topical, parenteral, intravenous, intra-arterial, subcutaneous, intramuscular, intracranial, intraorbital, ophthalmic, intraventricular, intracapsular, intraspinal, intracisternal, intraperitoneal, intranasal, aerosol, by suppositories, or oral administration.

This invention also includes pharmaceutical compositions which can contain one or more pharmaceutically acceptable carriers. In making the pharmaceutical compositions of the invention, the active ingredient is typically mixed with an excipient, diluted by an excipient or enclosed within such a carrier in the form of, for example, a capsule, sachet, paper, or other container. When the excipient serves as a diluent, it can be a solid, semisolid, or liquid material (e.g., normal saline), which acts as a vehicle, carrier or medium for the active ingredient. Thus, the compositions can be in the form of tablets, powders, lozenges, sachets, cachets, elixirs, suspensions, emulsions, solutions, syrups, and soft and hard gelatin capsules. As is known in the art, the type of diluent can vary depending upon the intended route of administration. The resulting compositions can include additional agents, such as preservatives.

The therapeutic agents of the invention can be administered alone, or in a mixture, in the presence of a pharmaceutically acceptable excipient or carrier. The excipient or carrier is selected on the basis of the mode and route of administration. Suitable pharmaceutical carriers, as well as pharmaceutical necessities for use in pharmaceutical formulations, are described in Remington: The Science and Practice of Pharmacy, 22^{nd} Ed., Gennaro, Ed., Lippencott Williams & Wilkins (2012), a well-known reference text in this field, and in the USP/NF (United States Pharmacopeia and the National Formulary), each of which is incorporated by reference. In preparing a formulation, the active compound can be milled to provide the appropriate particle size prior to combining with the other ingredients. If the active compound is substantially insoluble, it can be milled to a particle size of less than 200 mesh. If the active compound is substantially water soluble, the particle size can be adjusted by milling to provide a substantially uniform distribution in the formulation, e.g. about 40 mesh.

Examples of suitable excipients are lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, water, syrup, and methyl cellulose. The formulations can additionally include: lubricating agents such as talc, magnesium stearate, and mineral oil; wetting agents; emulsifying and suspending agents; preserving agents such as methyl- and propylhydroxy-benzoates; sweetening agents; and flavoring agents. Other exemplary excipients are described in Handbook of Pharmaceutical Excipients, 8th Edition, Sheskey et al., Eds., Pharmaceutical Press (2017), which is incorporated by reference.

The methods described herein can include the administration of a therapeutic, or prodrugs or pharmaceutical compositions thereof, or other therapeutic agents.

The pharmaceutical compositions can be formulated so as to provide immediate, extended, or delayed release of the active ingredient after administration to the patient by employing procedures known in the art.

The compositions can be formulated in a unit dosage form, each dosage containing, e.g., 0.1-500 mg of the active ingredient. For example, the dosages can contain from about 0.1 mg to about 50 mg, from about 0.1 mg to about 40 mg, from about 0.1 mg to about 20 mg, from about 0.1 mg to about 10 mg, from about 0.2 mg to about 20 mg, from about 0.3 mg to about 15 mg, from about 0.4 mg to about 10 mg, from about 0.5 mg to about 1 mg; from about 0.5 mg to about 100 mg, from about 0.5 mg to about 50 mg, from about 0.5 mg to about 30 mg, from about 0.5 mg to about 20 mg, from about 0.5 mg to about 10 mg, from about 0.5 mg to about 5 mg; from about 1 mg from to about 50 mg, from about 1 mg to about 30 mg, from about 1 mg to about 20 mg, from about 1 mg to about 10 mg, from about 1 mg to about 5 mg; from about 5 mg to about 50 mg, from about 5 mg to about 20 mg, from about 5 mg to about 10 mg; from about 10 mg to about 100 mg, from about 20 mg to about 200 mg, from about 30 mg to about 150 mg, from about 40 mg to about 100 mg, from about 50 mg to about 100 mg of the active ingredient, from about 50 mg to about 300 mg, from about 50 mg to about 250 mg, from about 100 mg to about 300 mg, or, from about 100 mg to about 250 mg of the active ingredient. For preparing solid compositions such as tablets, the principal active ingredient is mixed with one or more pharmaceutical excipients to form a solid bulk formulation composition containing a homogeneous mixture of a compound of the present invention. When referring to these bulk formulation compositions as homogeneous, the active ingredient is typically dispersed evenly throughout the composition so that the composition can be readily subdivided into equally effective unit dosage forms such as tablets and capsules. This solid bulk formulation is then subdivided into unit dosage forms of the type described above containing from, for example, 0.1 to about 500 mg of the active ingredient of the present invention.

Compositions for Oral Administration

The pharmaceutical compositions contemplated by the invention include those formulated for oral administration (“oral dosage forms”). Oral dosage forms can be, for example, in the form of tablets, capsules, a liquid solution or suspension, a powder, or liquid or solid crystals, which contain the active ingredient(s) in a mixture with non-toxic pharmaceutically acceptable excipients. These excipients may be, for example, inert diluents or fillers (e.g., sucrose, sorbitol, sugar, mannitol, microcrystalline cellulose, starches including potato starch, calcium carbonate, sodium chloride, lactose, calcium phosphate, calcium sulfate, or sodium phosphate); granulating and disintegrating agents (e.g., cellulose derivatives including microcrystalline cellulose, starches including potato starch, croscarmellose sodium, alginates, or alginic acid); binding agents (e.g., sucrose, glucose, sorbitol, acacia, alginic acid, sodium alginate, gelatin, starch, pregelatinized starch, microcrystalline cellulose, magnesium aluminum silicate, carboxymethylcellulose sodium, methylcellulose, hydroxypropyl methylcellulose, ethylcellulose, polyvinylpyrrolidone, or polyethylene glycol); and lubricating agents, glidants, and antiadhesives (e.g., magnesium stearate, zinc stearate, stearic acid, silicas, hydrogenated vegetable oils, or talc). Other pharmaceutically acceptable excipients can be colorants, flavoring agents, plasticizers, humectants, buffering agents, and the like.

Formulations for oral administration may also be presented as chewable tablets, as hard gelatin capsules wherein the active ingredient is mixed with an inert solid diluent (e.g., potato starch, lactose, microcrystalline cellulose, calcium carbonate, calcium phosphate or kaolin), or as soft gelatin capsules wherein the active ingredient is mixed with water or an oil medium, for example, peanut oil, liquid paraffin, or olive oil. Powders, granulates, and pellets may be prepared using the ingredients mentioned above under tablets and capsules in a conventional manner using, e.g., a mixer, a fluid bed apparatus or a spray drying equipment.

Controlled release compositions for oral use may be constructed to release the active drug by controlling the dissolution and/or the diffusion of the active drug substance. Any of a number of strategies can be pursued in order to obtain controlled release and the targeted plasma concentration vs time profile. In one example, controlled release is obtained by appropriate selection of various formulation parameters and ingredients, including, e.g., various types of controlled release compositions and coatings. Thus, the drug is formulated with appropriate excipients into a pharmaceutical composition that, upon administration, releases the drug in a controlled manner. Examples include single or multiple unit tablet or capsule compositions, oil solutions, suspensions, emulsions, microcapsules, microspheres, nanoparticles, patches, and liposomes. In certain embodiments, compositions include biodegradable, pH, and/or temperature-sensitive polymer coatings.

Dissolution or diffusion controlled release can be achieved by appropriate coating of a tablet, capsule, pellet, or granulate formulation of compounds, or by incorporating the compound into an appropriate matrix. A controlled release coating may include one or more of the coating substances mentioned above and/or, e.g., shellac, beeswax, glycowax, castor wax, carnauba wax, stearyl alcohol, glyceryl monostearate, glyceryl distearate, glycerol palm isostearate, ethylcellulose, acrylic resins, dl-polylactic acid, cellulose acetate butyrate, polyvinyl chloride, polyvinyl acetate, vinyl pyrrolidone, polyethylene, polymethacrylate, methylmethacrylate, 2-hydroxymethacrylate, methacrylate hydrogels, 1,3 butylene glycol, ethylene glycol methacrylate, and/or polyethylene glycols. In a controlled release matrix formulation, the matrix material may also include, e.g., hydrated methylcellulose, carnauba wax and stearyl alcohol, carbopol 934, silicone, glyceryl tristearate, methyl acrylate-methyl methacrylate, polyvinyl chloride, polyethylene, and/or halogenated fluorocarbon.

The liquid forms in which the compounds and compositions of the present invention can be incorporated for administration orally include aqueous solutions, suitably flavored syrups, aqueous or oil suspensions, and flavored emulsions with edible oils such as cottonseed oil, sesame oil, coconut oil, or peanut oil, as well as elixirs and similar pharmaceutical vehicles.

Compositions suitable for oral mucosal administration (e.g., buccal or sublingual administration) include tablets, lozenges, and pastilles, where the active ingredient is formulated with a carrier, such as sugar, acacia, tragacanth, or gelatin and glycerine.

Coatings

The pharmaceutical compositions formulated for oral delivery, such as tablets or capsules of the present invention can be coated or otherwise compounded to provide a dosage form affording the advantage of delayed or extended release. The coating may be adapted to release the active drug substance in a predetermined pattern (e.g., in order to achieve a controlled release formulation) or it may be adapted not to release the active drug substance until after passage of the stomach, e.g., by use of an enteric coating (e.g., polymers that are pH-sensitive (“pH controlled release”), polymers with a slow or pH-dependent rate of swelling, dissolution or erosion (“time-controlled release”), polymers that are degraded by enzymes (“enzyme-controlled release” or “biodegradable release”) and polymers that form firm layers that are destroyed by an increase in pressure (“pressure-controlled release”)). Exemplary enteric coatings that can be used in the pharmaceutical compositions described herein include sugar coatings, film coatings (e.g., based on hydroxypropyl methylcellulose, methylcellulose, methyl hydroxyethylcellulose, hydroxypropylcellulose, carboxymethylcellulose, acrylate copolymers, polyethylene glycols and/or polyvinylpyrrolidone), or coatings based on methacrylic acid copolymer, cellulose acetate phthalate, hydroxypropyl methylcellulose phthalate, hydroxypropyl methylcellulose acetate succinate, polyvinyl acetate phthalate, shellac, and/or ethylcellulose. Furthermore, a time delay material such as, for example, glyceryl monostearate or glyceryl distearate, may be employed.

For example, the tablet or capsule can comprise an inner dosage and an outer dosage component, the latter being in the form of an envelope over the former. The two components can be separated by an enteric layer which serves to resist disintegration in the stomach and permit the inner component to pass intact into the duodenum or to be delayed in release.

When an enteric coating is used, desirably, a substantial amount of the drug is released in the lower gastrointestinal tract.

In addition to coatings that effect delayed or extended release, the solid tablet compositions may include a coating adapted to protect the composition from unwanted chemical changes (e.g., chemical degradation prior to the release of the active drug substance). The coating may be applied on the solid dosage form in a similar manner as that described in Encyclopedia of Pharmaceutical Technology, vols. 5 and 6, Eds. Swarbrick and Boyland, 2000.

### Parenteral Administration

Within the scope of the present invention are also parenteral depot systems from biodegradable polymers. These systems are injected or implanted into the muscle or subcutaneous tissue and release the incorporated drug over extended periods of time, ranging from several days to several months. Both the characteristics of the polymer and the structure of the device can control the release kinetics which can be either continuous or pulsatile. Polymer-based parenteral depot systems can be classified as implants or microparticles. The former are cylindrical devices injected into the subcutaneous tissue whereas the latter are defined as spherical particles in the range of 10-100 μm. Extrusion, compression or injection molding are used to manufacture implants whereas for microparticles, the phase separation method, the spray-drying technique and the water-in-oil-in-water emulsion techniques are frequently employed. The most commonly used biodegradable polymers to form microparticles are polyesters from lactic and/or glycolic acid, e.g. poly(glycolic acid) and poly(L-lactic acid) (PLG/PLA microspheres). Of particular interest are in situ forming depot systems, such as thermoplastic pastes and gelling systems formed by solidification, by cooling, or due to the sol-gel transition, cross-linking systems and organogels formed by amphiphilic lipids. Examples of thermosensitive polymers used in the aforementioned systems include, N-isopropylacrylamide, poloxamers (ethylene oxide and propylene oxide block copolymers, such as poloxamer 188 and 407), poly(N-vinyl caprolactam), poly(siloethylene glycol), polyphosphazenes derivatives and PLGA-PEG-PLGA.

Mucosal Drug Delivery

Mucosal drug delivery (e.g., drug delivery via the mucosal linings of the nasal, rectal, vaginal, ocular, or oral cavities) can also be used in the methods described herein. Methods for oral mucosal drug delivery include sublingual administration (via mucosal membranes lining the floor of the mouth), buccal administration (via mucosal membranes lining the cheeks), and local delivery (Harris et al., Journal of Pharmaceutical Sciences, 81(1): 1-10, 1992).

Oral transmucosal absorption is generally rapid because of the rich vascular supply to the mucosa and allows for a rapid rise in blood concentrations of the therapeutic.

For buccal administration, the compositions may take the form of, e.g., tablets, lozenges, etc. formulated in a conventional manner. Permeation enhancers can also be used in buccal drug delivery. Exemplary enhancers include 23-lauryl ether, aprotinin, azone, benzalkonium chloride, cetylpyridinium chloride, cetyltrimethylammonium bromide, cyclodextrin, dextran sulfate, lauric acid, lysophosphatidylcholine, methol, methoxysalicylate, methyloleate, oleic acid, phosphatidylcholine, polyoxyethylene, polysorbate 80, sodium EDTA, sodium glycholate, sodium glycodeoxycholate, sodium lauryl sulfate, sodium salicylate, sodium taurocholate, sodium taurodeoxycholate, sulfoxides, and alkyl glycosides. Bioadhesive polymers have extensively been employed in buccal drug delivery systems and include cyanoacrylate, polyacrylic acid, hydroxypropyl methylcellulose, and poly methacrylate polymers, as well as hyaluronic acid and chitosan.

Liquid drug formulations (e.g., suitable for use with nebulizers and liquid spray devices and electrohydrodynamic (EHD) aerosol devices) can also be used. Other methods of formulating liquid drug solutions or suspension suitable for use in aerosol devices are known to those of skill in the art (see, e.g., Biesalski, U.S. Pat. No. 5,112,598, and Biesalski, U.S. Pat. No. 5,556,611).

Formulations for sublingual administration can also be used, including powders and aerosol formulations. Exemplary formulations include rapidly disintegrating tablets and liquid-filled soft gelatin capsules.

Dosing Regimes

The amount and frequency of administration of the compositions can vary depending on, for example, what is being administered, the state of the patient, and the manner of administration. In therapeutic applications, compositions can be administered to a patient suffering from the disease or condition in an amount sufficient to relieve or least partially relieve the symptoms of the disease or condition and its complications. The dosage is likely to depend on such variables as the type and extent of progression of the disease or condition, the severity of the disease or condition, the age, weight and general condition of the particular patient, the relative biological efficacy of the composition selected, formulation of the excipient, the route of administration, and the judgment of the attending clinician. Effective doses can be extrapolated from dose-response curves derived from in vitro or animal model test system. An effective dose is a dose that produces a desirable clinical outcome by, for example, improving a sign or symptom of the disease or condition or slowing its progression.

The amount of therapeutic per dose can vary. For example, a subject can receive from about 0.1 μg/kg to about 10,000 μg/kg. Generally, the therapeutic is administered in an amount such that the peak plasma concentration ranges from 150 nM-250 μM.

Exemplary dosage amounts can fall between 0.1-5000 μg/kg, 100-1500 μg/kg, 100-350 μg/kg, 340-750 μg/kg, or 750-1000 μg/kg. Exemplary dosages can 0.25, 0.5, 0.75, 1°, or 2 mg/kg. In another embodiment, the administered dosage can range from 0.05-5 mmol of therapeutic (e.g., 0.089-3.9 mmol) or 0.1-50 μmol of therapeutic (e.g., 0.1-25 μmol or 0.4-20 μmol).

The plasma concentration of therapeutic can also be measured according to methods known in the art. Exemplary peak plasma concentrations of therapeutic can range from 0.05-10 μM, 0.1-10 μM, 0.1-5.0 μM, or 0.1-1 μM. Alternatively, the average plasma levels of therapeutic can range from 400-1200 μM (e.g., between 500-1000 μM) or between 50-250 μM (e.g., between 40-200 μM). In some embodiments where sustained release of the drug is desirable, the peak plasma concentrations (e.g., of therapeutic) may be maintained for 6-14 hours, e.g., for 6-12 or 6-10 hours. In other embodiments where immediate release of the drug is desirable, the peak plasma concentration (e.g., of therapeutic) may be maintained for, e.g., 30 minutes.

The frequency of treatment may also vary. The subject can be treated one or more times per day with therapeutic (e.g., once, twice, three, four or more times) or every so-many hours (e.g., about every 2, 4, 6, 8, 12, or 24 hours). Preferably, the pharmaceutical composition is administered 1 or 2 times per 24 hours. The time course of treatment may be of varying duration, e.g., for two, three, four, five, six, seven, eight, nine, ten or more days. For example, the treatment can be twice a day for three days, twice a day for seven days, twice a day for ten days. Treatment cycles can be repeated at intervals, for example weekly, bimonthly or monthly, which are separated by periods in which no treatment is given. The treatment can be a single treatment or can last as long as the life span of the subject (e.g., many years).

Kits

Any of the pharmaceutical compositions of the invention described herein can be used together with a set of instructions, i.e., to form a kit. The kit may include instructions for use of the pharmaceutical compositions as a therapy as described herein. For example, the instructions may provide dosing and therapeutic regimes for use of the compounds of the invention to reduce symptoms and/or underlying cause of the disease or condition treated.

Table 1. Sunflower trypsin inhibitor (SFTI) template-based grafted peptides designed based on compound 18. Single letter amino acid code was used for peptide sequence. Lower case letters refer to D amino acids and capital letters refer to L amino acids. Chirality of Anapa was indicated with R and S configuration in italics. Antiproliferative activity of peptides in cancer cell lines are given as IC₅₀ in μM. Disulfide bonds between the cysteines are underlined.

| | | IC50 (μM) | | | | |
|---|---|---|---|---|---|---|
| | | BT-474 | MCF-7 | Calu-3 | A549 | MCF- |
| Code | Compound | (HER2+) | (HER2−) | (HER2+) | (HER2+) | 10A |
| Compound | Cyclo(PpR(R-X) | 0.197 ± 0.055 | >50 | 0.018 ± 0.013 | ND | |
| 18 | FDDF(R-X)R) | | | | | |
| SFTI-G1 | Cyclo(C(S-X)RDR | 4.16 ± 0.179 | >50 | ND | ND | |
| | (S-X)CFDSIDF) | | | | | |
| SFTI-G2 | Cyclo(C(R-X)RPpR | 5.23 ± 1.38 | >50 | ND | ND | |
| | (R-X)CFF) | | | | | |
| SFTI-G3 | Cyclo(C(R-X)RPPR | 3.65 ± 0.410 | >50 | ND | ND | |
| | (R-X)CF) | | | | | |
| SFTI-G4 | Cyclo(C(S-X)-RPGR | 17.22 ± 1.034 | >30 | ND | ND | |
| | (S-X)CFDSIPPDF) | | | | | |
| SFTI-G5 | Cyclo(C(S-X)RIPPR | 0.28 ± 0.021 | 25.61 ± 1.46 | 0.073 ± 0.0094 | 0.369 ± 0.07 | >50 |
| | (S-X)CFPDDF) | | | | | |
| SFTI-G6 | Cyclo(C(R-X)RIPPR | 2.94 ± 1.057 | >50 | ND | ND | ND |
| | (R-X)CFPDDF) | | | | | |
| Control | H₂N-K(X)F-OH | >100 | >100 | >100 | >100 | |
| Control-G | Cyclo(C(S-X)AIPPA | | | | | |
| | (S-X)CAPDAF) | >50 | >50 | >50 | >50 | |

*ND = not determined, X = Anapa, 3-amino napthyl propionic acid, a beta amino acid.

The invention illustratively disclosed herein suitably may explicitly be practiced in the absence of any element which is not specifically disclosed herein. While various embodiments of the present invention have been described in detail, it is apparent that various modifications and alterations of those embodiments will occur to and be readily apparent those skilled in the art. However, it is to be expressly understood that such modifications and alterations are within the scope and spirit of the present invention, as set forth in the appended claims. Further, the invention(s) described herein is capable of other embodiments and of being practiced or of being carried out in various other related ways. In addition, it is to be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items while only the terms “consisting of” and “consisting only of” are to be construed in the limitative sense.

Wherefore, I/we claim:

1. A method of treating an autoimmune or inflammatory disease comprising:
administering to the patient a pharmacologically effective amount of a therapeutic;
wherein the therapeutic includes one or more functional groups of a CD58-targeted peptide grafted onto a cyclic scaffold

2. The method of claim 1 one or more of the functional groups of a CD58-targeted peptide bind to CD58.

3. The method of claim 1 wherein the scaffold is one of a sunflower trypsin inhibitor (SFTI) and theta-defensins.

4. The method of claim 1 wherein the targeted peptide is chosen from the group of:
Peptide 6
Cyclo(SIYDpPDDIK)
SFTI-a
Cyclo(CKASAPPSCYDGDD)
SFTI-a1
Cyclo(CKSAPPSCAYDGDD)
SFTI-DBF
Cyclo(CKSA-DBF-SCAYDGDD)
SFTI-wt (control)
Cyclo(CTKSIPPICFPDGR)
Control
KGKTDAISVKAI-NH2;
where single letter code is used for amino acid representation, capital letters refer to L-amino acid, small letter refers to D-amino acid (P is L-proline, p is D-proline), cyclo indicates a cyclic compound, DBF is dibenzofuran, disulfide bond is indicated by underline.

5. The method of claim 4, wherein a peptide sequence of the targeted peptide one of freely changes conformations and is locked into beta conformation.

6. The method of claim 5, wherein the peptide sequence is locked into beta conformation through one of incorporation of 1,4 substituted triazole and substitution of Pro-Pro with dibenzofuran.

7. The method of claim 1 wherein the immune disease is one of rheumatoid arthritis, psoriatic arthritis, plaque psoriasis, ankylosing spondylitis, Crohn's disease and ulcerative colitis.

8. The method of claim 1 wherein cells associated with the immune disease are CD2-positive.

9. The method of claim 1 wherein cells associated with the immune disease are CD58-positive.

10. The method of claim 1 further comprising including a pharmacologically effective amount of one or more immune therapy agents.

11. The method of claim 10 wherein the immune therapy agents are one of etanercept, adalimumab, infliximab, certolizumab pegol, and golimumab.

12. The method of claim 1 wherein an administration route is one of oral, intravenous, and inhalation.

13. The method of claim 1 wherein the CD58-targeted peptide is administered with pharmaceutically acceptable excipients.
